(19) United States
(12) Patent Application Publication
(10) Pub. No.: US 2005/0170440 A1
(43) Pub. Date: Aug. 4, 2005

(54) Treatment and diagnosis of insulin-resistant states

(75) Inventors: Venita I. DeAlmeida, San Carlos, CA (US); Timothy A. Stewart, San Francisco, CA (US)

Correspondence Address: MERCHANT & GOULD PC, P.O. BOX 2903, MINNEAPOLIS, MN, 55402-0903, US

(73) Assignee: Genentech, Inc., South San Francisco, CA (US)

(21) Appl. No.: 11/056,562

(22) Filed: Feb. 11, 2005

Related U.S. Application Data
(63) Continuation of application No. 10/271,628, filed on Oct. 15, 2002, now abandoned.
(60) Provisional application No. 60/329,947, filed on Oct. 15, 2001.

(51) Int. Cl. A61K 38/28; G01N 33/53; G01N 33/567; A61K 38/22; C07K 16/46; A61K 31/175

(52) U.S. Cl. 435/7.2; 514/12; 530/391.1; 514/3; 514/592

(57) ABSTRACT

Dickkopf-5 (Dkk-5) protein is administered in effective amounts to treat disorders involving insulin resistance, such as non-insulin-dependent diabetes mellitus (NIDDM) or obesity. Also provided is a method of diagnosing insulin resistance and related disorders using Dkk-5 as a measure, and kits for diagnosis and treatment, as well as hybridomas producing antibodies to Dkk-5 and preparations comprising Dkk-5.

### RELATED APPLICATIONS

This application is a non-provisional application filed under 37 CFR 1.53(b)(1), claiming priority under 35 USC 119(e) to provisional application No. 60/329,947, filed Oct. 15, 2001, the contents of which are incorporated herein by reference.

### BACKGROUND OF THE INVENTION

1. Field Of The Invention

The present invention provides for the diagnosis and treatment of disorders involving insulin resistance, such as non-insulin-dependent, or Type 2, diabetes mellitus and other insulin-resistant states, such as those associated with obesity and aging. More particularly, the present invention relates to the use of Dkk-5 in the treatment of an insulin-resistant disorder. Also, the invention relates particularly to methods using levels of Dkk-5 to diagnose the presence of an insulin-resistant disorder in an individual suspected of having insulin resistance or related disorders, especially non-insulin dependent diabetes mellitus.

2. Description of Related Art

Insulin resistance, defined as a smaller than expected biological response to a given dose of insulin, is a ubiquitous correlate of obesity. Indeed, many of the pathological consequences of obesity are thought to involve insulin resistance. These include hypertension, hyperlipidemia and, most notably, non-insulin dependent diabetes mellitus (NIDDM). Most NIDDM patients are obese, and a very central and early component in the development of NIDDM is insulin resistance (Moller et al., New Eng. J. Med., 325: 938 (1991)). It has been demonstrated that a post-receptor abnormality develops during the course of insulin resistance, in addition to the insulin receptor downregulation during the initial phases of this disease (Olefsky et al., in Diabetes Mellitus, Rifkin and Porte, Jr., Eds. (Elsevier Science Publishing Co., Inc., New York, ed. 4, 1990), pp. 121-153).

Several studies on glucose transport systems as potential sites for such a post-receptor defect have demonstrated that both the quantity and function of the insulin-sensitive glucose transporter (Glut4) is deficient in insulin-resistant states of rodents and humans (Garvey et al., Science, 245: 60 (1989); Sivitz et al., Nature, 340: 72 (1989); Berger et al., Nature, 340: 70 (1989); Kahn et al., J. Clin. Invest., 84: 404 (1989); Charron et al., J. Biol. Chem., 265: 7994 (1990); Dohm et al., Am. J. Physiol., 260: E459 (1991); Sinha et al., Diabetes, 40: 472 (1991); Friedman et al., J. Clin. Invest., 89: 701 (1992)). A lack of a normal pool of insulin-sensitive glucose transporters could theoretically render an individual insulin resistant (Olefsky et al., in Diabetes Mellitus, supra). However, some studies have failed to show downregulation of Glut4 in human NIDDM, especially in muscle, the major site of glucose disposal (Bell, Diabetes, 40: 413 (1990); Pederson et al., Diabetes, 39: 865 (1990); Handberg et al., Diabetologia, 33: 625 (1990); Garvey et al., Diabetes, 41: 465 (1992)).

Evidence from in vivo studies in animal models and clinical studies indicate that insulin resistance in Type II diabetes can result from alterations in expression and activity of intermediates in the insulin signal transduction pathway, alterations in the rate of insulin-stimulated glucose transport, or alterations in translocation of GLUT4 to the plasma membrane (Zierath et al., Diabetologia, 43: 821-835 (2000)). Evidence from animal studies suggests that insulin-signaling defects in muscle alter whole-body glucose homeostasis (Saad et al., J. Clin. Invest., 90: 1839-1849 (1992); Folli et al., J. Clin. Invest., 92: 1787-1794 (1993); Heydrick et al., J. Clin. Invest., 91: 1358-1366 (1993); Saad et al., J. Clin Invest, 92: 2065-2072 (1993); Heydrick et al., Am. J. Physiol., 268: E604-612 (1995)); and defects in intermediates in the insulin signaling cascade, including the IR, IRS-1, and PI 3-kinase, can lead to reduced glucose transport and reduced insulin-stimulated GLUT4 translocation in skeletal muscle from insulin-resistant and Type II diabetic subjects. In some examples, altered expression of IRS-1 (Saad et al., 1992, supra; Saad et al., 1993, supra; Goodyear et al., J. Clin. Invest., 95: 2195-2204 (1995)), PI 3-kinase (Anai et al., Diabetes, 47: 13-23 (1998)), or GSK-3 (Nikoulina et al., Diabetes, 49: 263-271 (2000)), or decreased levels of PKCθ (Chalfant et al., Endocrinology, 141: 2773-2778 (2000)), or PTP1B (Dadke et al., Biochem. Biophys. Res. Commun., 274: 583-589 (2000)) have been observed. Decreased phosphorylation of IR (Arner et al., Diabetologia, 30: 437-440 (1987); Maegawa et al., Diabetes, 44: 815-819 (1991); Saad et al., 1992, supra, Saad et al., 1993, supra, Goodyear et al., supra), IRS-1 (Saad et al., 1992, supra; Saad et al., 1993, supra; Goodyear et al., supra), and Akt (Krook et al., Diabetes, 47: 1281-1286 (1998)) has also been observed in skeletal muscle of some Type II diabetic subjects. Additionally, decreased activity of PI 3-kinase (Saad et al., 1992, supra; Heydrick et al., 1995, supra; Saad et al., 1993, supra; Goodyear et al., supra; Heydrick et al., 1993, supra; Folli et al., Acta Diabetol. 33: 185-192 (1996); Bjornholm et al., Diabetes, 46: 524-527 (1997); Andreelli et al., Diabetologia, 42: 358-364 (1999); Kim et al., J. Clin. Invest, 104: 733-741 (1999); Andreelli F, et al., Diabetologia, 43: 356-363 (2000); Krook et al., Diabetes, 49-284-292 (2000)) and increased activity of GSK-3 (Eldar-Finkelmani et al., Diabetes, 48: 1662-1666 (1999)), PKC (Avignon et al., Diabetes, 45: 1396-1404 (1996)), and PTP1B (Dadke et al., supra) have also been shown to be associated with Type II diabetes. Additionally, the distribution of PKC isoforms is altered in skeletal muscle from diabetic animals (Schmitz-Peiffer et al., Diabetes, 46: 169-178 (1997)), and the content of PKCα, PKCβ, PKCε, and PKCδ is increased in membrane fractions and decreased in cytosolic fractions of soleus muscle in the non-obese Goto-Kakizaki (GK) diabetic rat (Avignon et al., supra).

Abnormal subcellular localization of GLUT4 has been observed in skeletal muscle from insulin-resistant subjects with or without Type II diabetes (Vogt et al., Diabetologia, 35: 456463 (1992); Garvey et al., J. Clin. Invest., 101: 2377-2386 (1998)), suggesting that defects in GLUT4 trafficking and translocation may cause insulin resistance in skeletal muscle. In vivo and in vitro studies have demonstrated a reduced rate of insulin-stimulated glucose transport in skeletal muscle in some Type II diabetic subjects (Andreasson et al., Acta Physiol. Scand., 142: 255-260 (1991); Zierath et al., Diabetologia, 37: 270-277 (1994); Bonadonna et al., Diabetes, 45: 915-925 (1996)).

Although the diagnosis of symptomatic diabetes mellitus is not difficult, detection of asymptomatic disease can raise a number of problems. Diagnosis may usually be confirmed by the demonstration of fasting hyperglycemia. In borderline cases, the well-known glucose tolerance test is usually applied. Some evidence suggests, however, that the oral glucose tolerance test over-diagnoses diabetes to a considerable degree, probably because stress from a variety of sources (mediated through the release of the hormone epinephrine) can cause an abnormal response. In order to clarify these difficulties, the National Diabetes Data Group of the National Institutes of Health have recommended criteria for the diagnosis of diabetes following a challenge with oral glucose (National Diabetes Data Group: Classification and diagnosis of diabetes mellitus and other categories of glucose intolerance. Diabetes, 28: 1039 (1979)).

The frequency of diabetes mellitus in the general population is difficult to ascertain with certainty, but the disorder is believed to affect more than ten million Americans. Diabetes mellitus generally cannot be cured but only controlled. In recent years it has become apparent that there are a series of different syndromes included under the umbrella term “diabetes mellitus”. These syndromes differ both in clinical manifestations and in their pattern of inheritance. The term diabetes mellitus is considered to apply to a series of hyperglycemic states that exhibit the characteristics noted above and below.

Diabetes mellitus has been classified into two basic categories, primary and secondary, and includes impaired glucose tolerance, which may be defined as a state associated with abnormally elevated blood glucose levels after an oral glucose load, in which the degree of elevation is insufficient to allow a diagnosis of diabetes to be made. Persons in this category are at increased risk for the development of fasting hyperglycemia or symptomatic diabetes relative to persons with normal glucose tolerance, although such a progression cannot be predicted in individual patients. In fact, several large studies suggest that most patients with impaired glucose tolerance (approximately 75 percent) never develop diabetes (Jarrett et al., Diabetologia, 16: 25-30 (1979)).

The independent risk factors obesity and hypertension for atherosclerotic diseases are also associated with insulin resistance. Using a combination of insulin/glucose clamps, tracer glucose infusion and indirect calorimetry, it has been demonstrated that the insulin resistance of essential hypertension is located in peripheral tissues (principally muscle) and correlates directly with the severity of hypertension (DeFronzo and Ferrannini, Diabetes Care 14: 173 (1991)). In hypertension of the obese, insulin resistance generates hyperinsulinemia, which is recruited as a mechanism to limit further weight gain via thermogenesis, but insulin also increases renal sodium reabsorption and stimulates the sympathetic nervous system in kidneys, heart, and vasculature, creating hypertension.

It is now appreciated that insulin resistance is usually the result of a defect in the insulin receptor signaling system, at a site post binding of insulin to the receptor. Accumulated scientific evidence demonstrating insulin resistance in the major tissues that respond to insulin (muscle, liver, adipose) strongly suggests that a defect in insulin signal transduction resides at an early step in this cascade, specifically at the insulin receptor kinase activity, which appears to be diminished (Haring, Diabetalogia, 34: 848 (1991)).

It is noteworthy that, notwithstanding other avenues of treatment, insulin therapy remains the treatment of choice for many patients with Type 2 diabetes, especially those who have undergone primary diet failure and are not obese, or those who have undergone both primary diet failure and secondary oral hypoglycemic failure. But it is equally clear that insulin therapy must be combined with a continued effort at dietary control and lifestyle modification, and in no way can be thought of as a substitute for these. In order to achieve optimal results, insulin therapy should be followed with self-blood glucose monitoring and appropriate estimates of glycosylated blood proteins: Insulin may be administered in various regimens alone, two or multiple injections of short, intermediate or long-acting insulins, or mixtures of more than one type. The best regimen for any patient must be determined by a process of tailoring the insulin therapy to the individual patient's monitored response.

The trend to the use of insulin therapy in Type 2 diabetes has increased with the modern realization of the importance of strict glycemic control in the avoidance of long-term diabetic complications. In non-obese Type 2 diabetics with secondary oral hypoglycemic failure, however, although insulin therapy may be successful in producing adequate control, a good response is by no means assured (Rendell et al., Ann. Int. Med., 90: 195-197 (1979)). In one study, only 31 percent of 58 non-obese patients who were poorly controlled on maximal doses of oral hypoglycemic agents achieved objectively verifiable improvement in control on a simple insulin regimen (Peacock et al., Br. Med. J., 288: 1958-1959 (1984)). In obese diabetics with secondary failure, the picture is even less clear-cut because in this situation insulin frequently increases body weight, often with a concomitant deterioration in control.

It will be apparent, therefore, that the current state of knowledge and practice with respect to the therapy of Type 2 diabetes is by no means satisfactory. The majority of patients undergo primary dietary failure with time, and the majority of obese Type 2 diabetics fail to achieve ideal body weight. Although oral hypoglycemic agents are frequently successful in reducing the degree of glycemia in the event of primary dietary failure, many authorities doubt that the degree of glycemic control attained is sufficient to avoid the occurrence of the long-term complications of atheromatous disease, neuropathy, nephropathy, retinopathy, and peripheral vascular disease associated with longstanding Type 2 diabetes. The reason for this can be appreciated in the light of the current realization that even minimal glucose intolerance, approximately equivalent to a fasting plasma glucose of 5.5 to 6.0 mmol/L, is associated with an increased risk of cardiovascular mortality (Fuller et al., Lancet, 1: 1373-1378 (1980)). It is also not clear that insulin therapy produces any improvement in long-term outcome over treatment with oral hypoglycemic agents. Thus, it can be appreciated that a superior method of treatment would be of great utility.

The Dickkopf (dkk) family of proteins is a family of secreted Wnt inhibitors (Krupnik et al., Gene, 238: 301-313 (1999); Monaghan et al., Mech Dev., 87: 45-56 (1999)). Dkk-1 (WO 00/12708 published Mar. 9, 2000, wherein the Dkk-1 is designated as PRO1316 and the encoding DNA as DNA60608) was identified as an inducer of head formation in Xenopus by inhibition of Wnt signaling (Glinka et al., Nature 391: 357-362 (1998)), and subsequently shown to be involved in limb development (Grotewold et al., Mech. Dev., 89: 151-153 (1999)) and inhibitory to Wnt-induced morphological transformation (Fedi et al., J. Biol. Chem., 274: 19465-19472 (1999)). It has been found that Dkk-1 and Dkk-2 exhibit mutual antagonism, in that Dkk-2 activates rather than inhibits the Wnt/β-catenin signaling pathway in Xenopus embryos (Wu et al., Current Biology, 10: 1611-1614 (2000)). It has also been reported that while Dkk-1 inhibits Wnt signaling, a cleavage product of Dkk-1 activates it (Brott and Sokol, Mol. Cell. Biol, 22: 6100-6110 (2000)).

Recent studies indicate that Dkks act by binding to the low-density lipoprotein related-protein LRP6, which acts as a co-receptor for Wnt signaling (Pinson et al., Nature, 407: 535-538 (2000); Tamai et al., Nature, 407: 530-535 (2000); Wehrli et al., Nature, 407: 527-530 (2000)). Dkk-1 antagonizes Wnt signaling by binding to LRP6 at domains distinct from those involved in its interaction with Wnt and Frizzled, thus inhibiting LRP6-mediated Wnt/β-catenin signaling (Bafico et al., Nat. Cell. Biol., 3: 683-686 (2001), Mao et al., Nature 411: 321-325 (2001); Semenov et al., Current Biology, 11: 951-961 (2001)).

The Wnt signaling pathway plays a key role in embryonic development, differentiation of various cell types, and oncogenesis (Peifer and Polakis, Science, 287: 1606-1609 (2000)). The Wnt signaling pathway is activated by the interaction between secreted Wnts and their receptors, the frizzled proteins (Hisken and Behrens, J. Cell Sci., 113: 3545-3546 (2000)). It leads to the activation of Disheveled (Dv11) protein, which activates Akt, which is subsequently recruited to Axin-β-catenin-GSK3βAPC (Fukumoto et al., J. Biol. Chem., 276: 17479-17483 (2001)). This is followed by the phosphorylation and inactivation of GSK3β, resulting in inhibition of the phosphorylation and degradation of β-catenin. The accumulated β-catenin is translocated to the nucleus where it interacts with transcription factors of the lymphoid enhancer factor-T cell factor (LEF/TCF) family and induces the transcription of target genes.

Two of the downstream effectors of Wnt signaling, Akt and GSK3β, are key intermediates in the insulin signaling pathway/glucose metabolism. Wnt signaling is involved in the regulation of muscle differentiation (Borello et al., Development, 126: 42474255 (1999); Cook et al., EMBO J. 15: 45264536 (1996); Cossu and Borello, EMBO J., 18: 6867-6872 (1999); Ridgeway et al., J. Biol. Chem., 275: 32398-32405 (2000); Tian et al., Development, 126: 3371-3380 (1999); Toyofuku et al., J. Cell. Biol., 150: 225-241 (2000)) and adipogenesis (Ross et al., Science, 289: 950-953 (2000)). Inhibition of Wnt signaling can stimulate the trans-differentiation of myocytes to adipocytes (Ross et al., supra). In addition, LRP5 is genetically associated with Type 1 diabetes. The gene is within the insulin-dependent diabetes mellitus (IDDM) locus IDDM4 on chromosome 11q13 (Hey et al., Gene, 216: 103-111 (1998)) and is expressed in the islets of Langerhans, macrophages, and Vitamin A system cells, which are cell types that are involved in the progression of Type I diabetes. (Figueroa et al., J. Histochem. Cytochem., 48: 1357-1368 (2000)). LRP5 mRNA was increased in the liver and accumulated in cholesterol-laden foam cells of atherosclerotic lesions in LDLR-deficient Watanabe heritable hyperlipidemic rabbits (Kim et al., J. Biochem. (Tokyo), 124: 1072-1076 (1998)).

A Dkk-5 molecule is described in WO 01/40465 (PCT/US00/30873), wherein the Dkk-5 is designated as PRO10268, and the encoding DNA as DNA145583-2820, with the ATCC deposit no. PTA-1179, deposited on Jan. 11, 2000. Another Dkk-5 molecule with an amino acid change in the mature region as compared to the molecule in WO 01/40465 is identified in EP 1067182-A2 published Jan. 10, 2001 (designated PSEC0258). The latter application relates to several nucleic acid sequences that encode human secretory or membrane proteins and antibodies thereto. The focus of their utility is contained in two examples. The first is treating NT cells with rheumatoid arthritis (RA) and RA inhibitors and looking at up/downregulation of a subset of the discovered genes as they go through neuronal differentiation. The second example involves treating primary cells from synovial tissue with TNF-alpha for RA and looking at the up/downregulation of a subset of their genes. In neither case is the Dkk-5 molecule of EP 1067182-A2 a positive hit.

There is a need for effective therapeutic agents that can be used in the diagnosis and therapy of individuals suffering from an insulin-resistant disorder, including NIDDM.

### SUMMARY OF THE INVENTION

The protein Dkk-5 was identified as a modulator of glucose metabolism in cultured skeletal muscle cells and adipocytes. Treatment of muscle cells with Dkk-5 resulted in an increase in the basal and insulin-stimulated glucose uptake. This effect was observed following long-term treatment, suggesting that Dkk-5 affects both muscle differentiation as well as the expression levels of proteins in the insulin-signaling pathway. The data show that Dkk-5 stimulates both basal and insulin-stimulated glucose metabolism in vitro. Hence, Dkk-5 is useful in the treatment of an insulin-resistant disorder, including one associated with, for example, obesity, glucose intolerance, diabetes mellitus, hypertension, and ischemic diseases of the large and small blood vessels.

The invention herein consists of the methods, kits, and compositions as claimed. Specifically, the invention provides in one embodiment a method of treating an insulin-resistant disorder in mammals comprising administering to a mammal in need thereof an effective amount of Dkk-5. Preferably, the mammal is human and has NIDDM or is obese. Also preferred is systemic administration. In a further preferred embodiment, another insulin-resistance-treating agent is administered in addition to the Dkk-5 to treat the disorder of insulin resistance.

In a still further preferred embodiment, the Dkk-5 polypeptide used for treatment has at least about 85%, more preferably at least about 90%, more preferably at least about 95%, more preferably at least about 99%, and most preferably 100% amino acid sequence identity to SEQ ID NO:5 in FIG. 2, with or without its associated signal peptide. In another preferred embodiment, the Dkk-5 is an internal cleavage protein fragment of SEQ ID NO:5 having N-terminal sequence MALFDWTDYEDLK (SEQ ID NO:8) and a molecular weight of about 16 kDa, or is a mixture of a Dkk-5 having SEQ ID NO:5 and an internal cleavage protein fragment of SEQ ID NO:5 having N-terminal sequence MALFDWTDYEDLK (SEQ ID NO:8) and a molecular weight of about 16 kDa, or is a mixture of a Dkk-5 having SEQ ID NO:5 lacking its associated signal peptide and an internal cleavage protein fragment of SEQ ID NO:5 having N-terminal sequence MALFDWTDYEDLK (SEQ ID NO:8) and a molecular weight of about 16 kDa. More preferably, the Dkk-5 is a Dkk-5 comprising SEQ ID NO:5, or a Dkk-5 comprising the sequence between residue 20 up to residue 30 and residue 347 (the end) of SEQ ID NO:5, preferably a Dkk-5 comprising the sequence between residues 25 and 347 of SEQ ID NO:5, or an internal cleavage protein fragment of SEQ ID NO:5 having N-terminal sequence MALFDWTDYEDLK (SEQ ID NO:8) and a molecular weight of about 16 kDa, or a combination of said cleavage product and one or both of the Dkk-5 comprising SEQ ID NO:5 or comprising the sequence between residue 20 up to residue 30 and residue 347 of SEQ ID NO:5.

In another embodiment of the invention a method is provided for detecting the presence or onset of an insulin-resistant disorder in a mammal. This method comprises the steps of:
(a) measuring the amount of Dkk-5 in a sample from said mammal; and
(b) comparing the amount determined in step (a) to an amount of Dkk-5 present in a standard sample, a decreased level in the amount of Dkk-5 in step (a) being indicative of the disorder. Preferably, the mammal is a human. Also, preferably the measuring is carried out using an anti-Dkk-5 antibody, such as a monoclonal antibody, in an immunoassay. Also, preferably such an anti-Dkk-5 antibody comprises a label, more preferably a fluorescent label, a radioactive label, or an enzyme label, such as a bioluminescent label or a chemiluminescent label. Also, preferably, the immunoassay is a radioimmunoassay, an enzyme immunoassay, an enzyme-linked immunosorbent assay, a sandwich immunoassay, a precipitation assay, an immunoradioactive assay, a fluorescence immunoassay, a protein A immunoassay, or an immunoelectrophoresis assay. Also preferred is the situation where the insulin-resistant disorder is NIDDM.

In another embodiment, the invention provides a diagnostic kit for detecting the presence or onset of an insulin-resistant disorder in a mammal, said kit comprising:
(a) a container comprising an antibody that binds Dkk-5;
(b) a container comprising a standard sample containing Dkk-5; and
(c) instructions for using the antibody and standard sample to detect the disorder in a sample from the mammal, wherein either the antibody that binds Dkk-5 is detectably labeled or the kit further comprises another container comprising a second antibody that is detectably labeled and binds to the Dkk-5 or to the antibody that binds Dkk-5. Preferably the antibody binding Dkk-5 is a monoclonal antibody and the mammal is a human.

In a further embodiment, the invention provides a kit for treating an insulin-resistant disorder in a mammal, said kit comprising:
(a) a container comprising Dkk-5; and
(b) instructions for using the Dkk-5 to treat the disorder.
   In a preferred embodiment, the disorder is NIDDM, the container is a vial, and the instructions specify placing the contents of the vial in a syringe for immediate injection. Also preferred is where the kit further comprises a container comprising an insulin-resistance-treating agent and where the mammal is a human.

In another embodiment, the invention provides an isolated internal cleavage protein fragment of SEQ ID NO:5 having N-terminal sequence MALFDWTDYEDLK (SEQ ID NO:8) and a molecular weight of about 16 kDa.

In a further aspect, the invention supplies a composition comprising this protein fragment and a carrier, and more preferably this composition further comprises a Dkk-5 comprising SEQ ID NO:5 with or lacking its associated signal peptide. If the Dkk-5 comprising SEQ ID NO:5 lacks its associated signal peptide, it generally comprises the sequence between about residue 20 up to about residue 30 to the end of SEQ ID NO:5, more preferably residues 25 to 347 of SEQ ID NO:5.

The invention further provides a hybridoma producing a Dkk-5 antibody selected from PTA-3090, PTA-3091, PTA-3092, PTA-3093, PTA-3094, PTA-3095, and PTA-3096. Also provided is an antibody produced by any one of these hybridomas.

The invention further provides a method of evaluating the effect of a candidate pharmaceutical drug on an insulin-resistant disorder in a mammal comprising administering said drug to a transgenic non-human animal model that overexpresses the dkk-5 cDNA and determining the effect of the drug on glucose clearance from the blood of said model. Preferably, the animal model is a rodent, more preferably a mouse or rat, and most preferably a mouse model. In another preferred embodiment, the dkk-5 cDNA overexpressed by the model is under the control of a muscle-specific promoter, and the cDNA is overexpressed in muscle tissue.

### BRIEF DESCRIPTION OF THE DRAWINGS

FIG. 1 discloses the schematic structure of the human Dkk family of proteins (hDkk-1, hDkk-2, hDkk-4, hDkk-3, and hDkk-5).

FIG. 2 denotes the sequence alignment of the human Dkk family of proteins, Dkk-1 (SEQ ID NO: 1), Dkk-2 (SEQ ID NO:2), Dkk-3 (SEQ ID NO:3), Dkk-4 (SEQ ID NO:4), and Dkk-5 (SEQ ID NO:5). The boxed regions denote the cysteine-rich domains, and the inverted triangles denote the location of the internal cleavage site for proteins in this family.

FIG. 3 shows the relative expression levels of Dkk-5 in various adult human tissues.

FIG. 4 shows the relative levels of Dkk-5 expression in the mouse embryo.

FIG. 5A-5E show in situ hybridization analysis of whole mouse embryos at different days of development, with FIG. 5A being day 8.5-9 p.c., FIG. 5B day 10 p.c., FIG. 5C day 10 (close-up) p.c., FIG. 5D day 11 p.c., and FIG. 5E day 12.5 (head) p.c.

FIG. 6 shows the relative expression level of Dkk-5 during L6 cell differentiation from day 1 to day 8. FIG. 7 shows a SDS-PAGE Coomassie blue stained gel of hDkk-5 expressed in baculovirus and its clipping, with lane 1 being non-reducing conditions and lane 2 being reducing conditions.

FIG. 8A-8B show the effect of Dkk-5 on basal and insulin-stimulated glucose uptake in L6 muscle cells at 48-hour treatment (FIG. 8A) and 96-hour treatment (FIG. 8B). The lower bars represent no insulin use and the higher bars represent use of 30 nM insulin.

FIG. 9A-9B show the effect of Dkk-5 on basal and insulin-stimulated incorporation of glucose into glycogen in L6 muscle cells at 48-hour treatment (FIG. 9A) and 96-hour treatment (FIG. 9B). The lower bars represent no insulin use and the higher bars represent use of 30 nM insulin.

FIGS. 10A-10G depict the effect of Dkk-5 on the expression levels of different genes involved in myogenesis in L6 muscle cells. FIG. 10A shows the effect on myosin light chain (MLC-2) expression; FIG. 10B shows the effect on Myf5 expression, FIG. 10C shows the effect on myogenin expression, FIG. 10D shows the effect on Pax3 expression; FIG. 10E shows the effect on MLC ⅓ expression; FIG. 10F shows the effect on MyoD expression; and FIG. 10G shows the effect on myosin heavy chain (HC) expression. The diamonds represent untreated cells and the triangles represent cells treated with Dkk-5.

FIG. 11 shows the effect of Dkk-5 on expression of genes involved in the insulin-signaling pathway (involved in glucose metabolism). The bar to the left in each pair is Dkk-5 on Day 5 and the bar to the right in each pair is Dkk-5 on day 7.

FIG. 12 shows a FACS analysis of binding to L6 cells of Dkk-5 and what can abolish the binding.

FIGS. 13A-13B show the effect of Dkk-5 on basal and insulin-stimulated glucose uptake in adipocytes at 48-hour treatment (FIG. 13A) and 96-hour treatment (FIG. 13B). The lower bars represent no insulin use and the higher bars represent use of 30 nM insulin.

FIGS. 14A-14B show the effect of Dkk-5 on basal and insulin-stimulated glucose incorporation into lipids in adipocytes at 48-hour treatment (FIG. 14A) and 96-hour treatment (FIG. 14B). The lower bars represent no insulin use and the higher bars represent use of 30 nM insulin.

### DETAILED DESCRIPTION OF THE PREFERRED EMBODIMENTS

Definitions

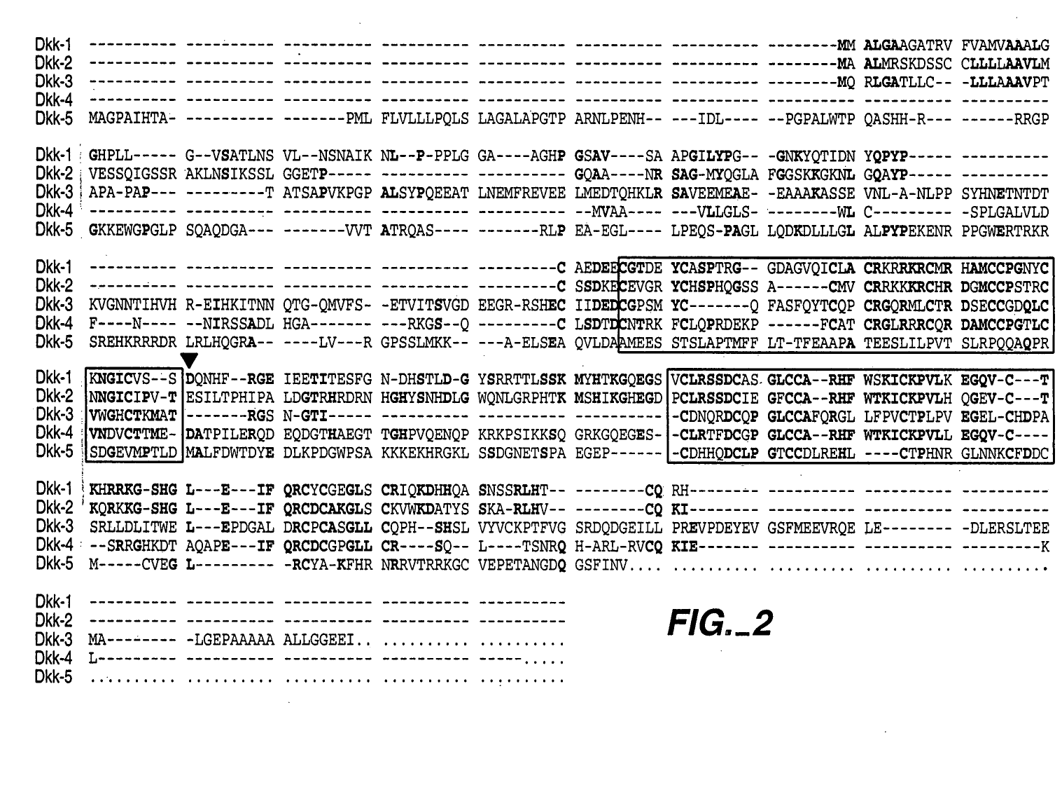

As used herein, “Dkk-5” or “Dickkopf-5” or “Dkk-5 polypeptide” refers to a polypeptide having at least about 80% amino acid sequence identity to the full-length amino acid sequence of the Dkk-5 polypeptide shown in FIG. 2 (SEQ ID NO:5), or a polypeptide having at least about 80% amino acid sequence identity to the amino acid sequence of the Dkk-5 polypeptide shown in FIG. 2 (SEQ ID NO:5) lacking its associated signal peptide, or a polypeptide having at least 80% amino acid sequence identity to an amino acid sequence encoded by the full-length coding sequence of the DNA deposited under ATCC accession number PTA-179, or any other fragment of full-length polypeptide SEQ ID NO:5 as disclosed herein, provided that the Dkk-5 polypeptide as defined herein has the activity of treating an insulin-resistant disorder.

The Dkk-5 defined herein may be isolated from a variety of sources, such as from human tissue types or from another native source, or prepared by recombinant or synthetic methods. The term “Dkk-5” specifically encompasses naturally-occurring truncated or secreted forms of the specific polypeptide (e.g., an extracellular domain sequence), naturally occurring variant forms (e.g., alternatively spliced forms) and naturally occurring allelic variants of the polypeptide. In various embodiments of the invention, the Dkk-5 polypeptide is a mature or full-length native sequence polypeptide comprising the full-length amino acid sequence of SEQ ID NO:5 shown in FIG. 2. However, while the Dkk-5 polypeptide disclosed in the accompanying FIG. 2 as SEQ ID NO:5 is shown to begin with a methionine residue, it is conceivable and possible that other methionine residues located either upstream or downstream from the beginning amino acid position of SEQ ID NO:5 in FIG. 2 may be employed as the starting amino acid residue for the Dkk-5 polypeptide.

Dkk-5 polypeptides include, for instance, polypeptides wherein one or more amino acid residues are added, or deleted, at the N- or C-terminus of the full-length native amino acid sequence of SEQ ID NO:5. A Dkk-5 polypeptide will have at least about 80% amino acid sequence identity, alternatively at least about 81% amino acid sequence identity, alternatively at least about 82% amino acid sequence identity, alternatively at least about 83% amino acid sequence identity, alternatively at least about 84% amino acid sequence identity, alternatively at least about 85% amino acid sequence identity, alternatively at least about 86% amino acid sequence identity, alternatively at least about 87% amino acid sequence identity, alternatively at least about 88% amino acid sequence identity, alternatively at least about 89% amino acid sequence identity, alternatively at least about 90% amino acid sequence identity, alternatively at least about 91% amino acid sequence identity, alternatively at least about 92% amino acid sequence identity, alternatively at least about 93% amino acid sequence identity, alternatively at least about 94% amino acid sequence identity, alternatively at least about 95% amino acid sequence identity, alternatively at least about 96% amino acid sequence identity, alternatively at least about 97% amino acid sequence identity, alternatively at least about 98% amino acid sequence identity, alternatively at least about 99% amino acid sequence identity, and alternatively 100% amino acid sequence identity to SEQ ID NO:5 as disclosed herein, or to SEQ ID NO:5 lacking the signal peptide as disclosed herein, provided it have the activity of treating an insulin-resistant disorder.

Ordinarily, the Dkk-5 polypeptides are at least about 10 amino acids in length, alternatively at least about 20 amino acids in length, alternatively at least about 30 amino acids in length, alternatively at least about 40 amino acids in length, alternatively at least about 50 amino acids in length, alternatively at least about 60 amino acids in length, alternatively at least about 70 amino acids in length, alternatively at least about 80 amino acids in length, alternatively at least about 90 amino acids in length, alternatively at least about 100 amino acids in length, alternatively at least about 150 amino acids in length, alternatively at least about 200 amino acids in length, alternatively at least about 300 amino acids in length, or more, provided it have the activity of treating an insulin-resistant disorder.

The isolated internal cleavage product (starting with MA) formed upon cleavage at the internal site marked by an inverted arrow in SEQ ID NO:5 of FIG. 2 having about 16 kDa molecular weight is active in enhancing basal and insulin-stimulated glucose uptake in muscle cells, just as is the recombinant preparation containing mostly the mature protein and/or signal-sequence-containing protein.

Preferred are those with at least about 85%, more preferably at least about 90%, more preferably at least about 95%, more preferably at least about 99% amino acid sequence identity to SEQ ID NO:5. More preferred still are the polypeptide of SEQ ID NO:5 of FIG. 2 herein, the polypeptide designated as PRO10268 in WO 01/40465 (PCT/US00/30873), and the polypeptide designated as PSEC0258 in EP 1067182-A2 published Jan. 10, 2001. Still more preferred are the polypeptide having SEQ ID NO:5 of FIG. 2 herein and PRO10268 of WO 01/40465 and the mature polypeptides therefrom, as well as the internal cleavage protein fragment of SEQ ID NO:5 having N-terminal sequence MALFDWTDYEDLK (SEQ ID NO:8) and a molecular weight of about 16 kDa and mixtures thereof with a Dkk-5 having SEQ ID NO:5 with or lacking its associated signal peptide. Most preferred is the polypeptide comprising SEQ ID NO:5 of FIG. 2 herein, with or without its associated signal peptide, and/or the internal cleavage protein fragment of SEQ ID NO:5 having N-terminal sequence MALFDWTDYEDLK (SEQ ID NO:8) and a molecular weight of about 16 kDa.

The approximate location of the “signal peptide” of the polypeptide disclosed herein is from the methionine at position 1 to the alanine at position 24 of SEQ ID NO:5 of FIG. 2, with the cleavage site being between the alanine at position 24 and the glycine at position 25 of SEQ ID NO:5 of FIG. 2. It is noted, however, that the C-terminal boundary of a signal peptide may vary, but most likely by no more than about five amino acids on either side of the signal peptide C-terminal boundary as initially identified herein, wherein the C-terminal boundary of the signal peptide may be identified pursuant to criteria routinely employed in the art for identifying that type of amino acid sequence element (e.g., Nielsen et al., Prot. Eng., 10: 1-6 (1997) and von Heinje et al., Nucl. Acids. Res., 14: 46834690 (1986)). Moreover, it is also recognized that, in some cases, cleavage of a signal sequence from a secreted polypeptide is not entirely uniform, resulting in more than one secreted species. These mature polypeptides, where the signal peptide is cleaved within no more than about five amino acids on either side of the C-terminal boundary of the signal peptide as identified herein, and the polynucleotides encoding them, are contemplated by the present invention.

“Percent (%) amino acid sequence identity” with respect to the Dkk-5 polypeptide sequences identified herein is defined as the percentage of amino acid residues in a candidate sequence that are identical with the amino acid residues in the specific polypeptide sequence, after aligning the sequences and introducing gaps, if necessary to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software, such as BLAST, BLAST-2, ALIGN, or Megalign (DNASTAR) software. Those skilled in the art can determine appropriate parameters for measuring alignment, including any algorithms needed to achieve maximal alignment over the full length of the sequences being compared. For purposes herein, however, % amino acid sequence identity values are generated using the sequence comparison computer program ALIGN-2, wherein the complete source code for the ALIGN-2 program is provided in Table 1 of WO01/16319 published Mar. 8, 2001 and WO00/73452 published Dec. 7, 2000. The ALIGN-2 sequence comparison computer program was authored by Genentech, Inc. and the source code has been filed with user documentation in the U.S. Copyright Office, Washington D.C., 20559, where it is registered under U.S. Copyright Registration No. TXU510087. The ALIGN-2 program is publicly available through Genentech, Inc., South San Francisco, Calif. The ALIGN-2 program should be compiled for use on a UNIX operating system, preferably digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:
100 times the fraction X/Y
where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Examples of calculations of amino acid sequence identities using ALIGN-2 are provided in Tables 2 and 3 of WO01/16319 published Mar. 8, 2001 and WO0/73452 published Dec. 7, 2000.

Unless specifically stated otherwise, all % amino acid sequence identity values used herein are obtained as described in the immediately preceding paragraph using the ALIGN-2 computer program. However, % amino acid sequence identity values may also be obtained as described below by using the WU-BLAST-2 computer program (Altschul et al., Methods in Enzymology, 266: 460-480 (1996)). Most of the WU-BLAST-2 search parameters are set to the default values. Those not set to default values, i.e., the adjustable parameters, are set with the following values: overlap span=1, overlap fraction=0.125, word threshold (T)=11, and scoring matrix=BLOSUM62. When WU-BLAST-2 is employed, a % amino acid sequence identity value is determined by dividing (a) the number of matching identical amino acid residues between the amino acid sequence of the Dkk-5 polypeptide of interest having a sequence derived from the native Dkk-5 polypeptide and the comparison amino acid sequence of interest (i.e., the sequence against which the Dkk-5 polypeptide of interest is being compared) as determined by WU-BLAST-2 by (b) the total number of amino acid residues of the Dkk-5 polypeptide of interest. For example, in the statement “a polypeptide comprising the amino acid sequence A which has or having at least 80% amino acid sequence identity to the amino acid sequence B”, the amino acid sequence A is the comparison amino acid sequence of interest and the amino acid sequence B is the amino acid sequence of the Dkk-5 polypeptide of interest.

Percent amino acid sequence identity may also be determined using the sequence comparison program NCBI-BLAST2 (Altschul et al., Nucleic Acids Res., 25: 3389-3402 (1997)). The NCBI-BLAST2 sequence comparison program may be downloaded from http://www.ncbi.nlm.nih gov or otherwise obtained from the National Institute of Health, Bethesda, Md. NCBI-BLAST2 uses several search parameters, wherein all of those search parameters are set to default values including, for example, unmask=yes, strand=all, expected occurrences=10, minimum low complexity length=15/5, multi-pass e-value=0.01, constant for multi-pass=25, dropoff for final gapped alignment=25 and scoring matrix=BLOSUM62.

In situations where NCBI-BLAST2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:
100 times the fraction X/Y
where X is the number of amino acid residues scored as identical matches by the sequence alignment program NCBI-BLAST2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A.

As used herein, “treating” describes the management and care of a patient for the purpose of combating an insulin-resistant disorder and includes the administration to prevent the onset of the symptoms or complications, alleviate the symptoms or complications, or eliminate the insulin-resistant disease, condition, or disorder. For purposes of this invention, beneficial or desired clinical results include, but are not limited to, alleviation of symptoms associated with insulin resistance, diminishment of the extent of the symptoms of insulin resistance, stabilization (i.e., not worsening) of the symptoms of insulin resistance (e.g., reduction of insulin requirement), increase in insulin sensitivity and/or insulin secretion to prevent islet cell failure, and delay or slowing of insulin-resistance progression, e.g., diabetes progression. As will be understood by one of skill in the art, the particular symptoms that yield to treatment in accordance with the invention will depend on the type of insulin-resistant disorder being treated. Those “in need of treatment” include mammals already having the disorder, as well as those prone to having the disorder, including those in which the disorder is to be prevented.

The term “mammal” for the purposes of treatment and diagnosis refers to any animal classified as a mammal, including but not limited to, humans, sport, zoo, pet, and domestic or farm animals, such as dogs, cats, cattle, sheep, pigs, horses, and primates, such as monkeys. Preferably the mammal is a human.

An “insulin-resistant disorder” is a disease, condition, or disorder resulting from a failure of the normal metabolic response of peripheral tissues (insensitivity) to the action of exogenous insulin, i.e., it is a condition where the presence of insulin produces a subnormal biological response. In clinical terms, insulin resistance is present when normal or elevated blood glucose levels persist in the face of normal or elevated levels of insulin. It represents, in essence, a glycogen synthesis inhibition, by which either basal or insulin-stimulated glycogen synthesis, or both, are reduced below normal levels. Insulin resistance plays a major role in Type 2 diabetes, as demonstrated by the fact that the hyperglycemia present in Type 2 diabetes can sometimes be reversed by diet or weight loss sufficient, apparently, to restore the sensitivity of peripheral tissues to insulin. The term includes abnormal glucose tolerance, as well as the many disorders in which insulin resistance plays a key role, such as obesity, diabetes mellitus, ovarian hyperandrogenism, and hypertension.

“Diabetes mellitus” refers to a state of chronic hyperglycemia, i.e., excess sugar in the blood, consequent upon a relative or absolute lack of insulin action. There are three basic types of diabetes mellitus, type I or insulin-dependent diabetes mellitus (IDDM), type II or non-insulin-dependent diabetes mellitus (NIDDM), and type A insulin resistance, although type A is relatively rare. Patients with either type I or type H diabetes can become insensitive to the effects of exogenous insulin through a variety of mechanisms. Type A insulin resistance results from either mutations in the insulin receptor gene or defects in post-receptor sites of action critical for glucose metabolism. Diabetic subjects can be easily recognized by the physician, and are characterized by hyperglycemia, impaired glucose tolerance, glycosylated hemoglobin and, in some instances, ketoacidosis associated with trauma or illness.

“Non-insulin dependent diabetes mellitus” or “NIDDM” refers to Type II diabetes. NIDDM patients have an abnormally high blood glucose concentration when fasting and delayed cellular uptake of glucose following meals or after a diagnostic test known as the glucose tolerance test. NIDDM is diagnosed based on recognized criteria (American Diabetes Association, Physician's Guide to Insulin-Dependent (Type I) Diabetes, 1988; American Diabetes Association, Physician's Guide to Non-Insulin-Dependent (Type II) Diabetes, 1988).

Symptoms and complications of diabetes to be treated as a disorder as defined herein include hyperglycemia, unsatisfactory glycemic control, ketoacidosis, insulin resistance, elevated growth hormone levels, elevated levels of glycosylated hemoglobin and advanced glycosylation end-products (AGE), dawn phenomenon, unsatisfactory lipid profile, vascular disease (e.g., atherosclerosis), microvascular disease, retinal disorders (e.g., proliferative diabetic retinopathy), renal disorders, neuropathy, complications of pregnancy (e.g., premature termination and birth defects) and the like. Included in the definition of treatment are such end points as, for example, increase in insulin sensitivity, reduction in insulin dosing while maintaining glycemic control, decrease in HbA1c, improved glycemic control, reduced vascular, renal, neural, retinal, and other diabetic complications, prevention or reduction of the “dawn phenomenon”, improved lipid profile, reduced complications of pregnancy, and reduced ketoacidosis.

A “therapeutic composition” or “composition,” as used herein, is defined as comprising Dkk-5 and a pharmaceutically acceptable carrier, such as water, minerals, proteins, and other excipients known to one skilled in the art.

The term “antibody” herein is used in the broadest sense and specifically covers intact monoclonal antibodies, polyclonal antibodies, multispecific antibodies (e.g. bispecific antibodies) formed from at least two intact antibodies, and antibody fragments, so long as they exhibit the desired biological activity as set forth herein, for example, binding to Dkk-5 in a diagnostic assay.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to polyclonal antibody preparations that include different antibodies directed against different determinants (epitopes), each monoclonal antibody is directed against a single determinant on the antigen.

In addition to their specificity, the monoclonal antibodies are advantageous in that they may be synthesized uncontaminated by other antibodies. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler and Milstein, Nature, 256: 495 (1975), or may be made by recombinant DNA methods (e.g., U.S. Pat. No. 4,816,567). The “monoclonal antibodies” may also be isolated from phage antibody libraries using the techniques described in Clackson et al., Nature, 352: 624-628 (1991) and Marks et al., J. Mol. Biol., 222: 581-597 (1991), for example.

The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity as noted herein (U.S. Pat. No. 4,816,567; Morrison et al., Proc. Natl. Acad. Sci. USA, 81: 6851-6855 (1984)). Chimeric antibodies of interest herein include “primatized” antibodies comprising variable domain antigen-binding sequences derived from a non-human primate (e.g. Old World Monkey, ape, etc.) and human constant-region sequences.

“Antibody fragments” comprise a portion of an intact antibody, preferably comprising the antigen-binding or variable region thereof. Examples of antibody fragments include Fab, Fab′, F(ab′)₂, and Fv fragments; diabodies; linear antibodies; single-chain antibody molecules; and multispecific antibodies formed from antibody fragment(s).

An “intact” antibody is one that comprises an antigen-binding variable region as well as a light-chain constant domain (C_{L}) and heavy-chain constant domains (C_{H}1, C_{H}2 and C_{H}3). The constant domains may be native-sequence constant domains (e.g., human native-sequence constant domains) or an amino acid sequence variant thereof.

The term “sample,” as used herein, refers to a biological sample containing or suspected of containing Dkk-5. This sample may come from any source, preferably a mammal and more preferably a human. Such samples include aqueous fluids, such as serum, plasma, lymph fluid, synovial fluid, follicular fluid, seminal fluid, milk whole blood, urine, cerebrospinal fluid, saliva, sputum, tears, perspiration, mucous, tissue culture medium, tissue extracts, and cellular extracts.

An “insulin-resistance-treating agent” or “hypoglycemic agent” (used interchangeably herein) is an agent other than Dkk-5 that is used to treat an insulin-resistant disorder, such as, e.g., insulin (one or more different insulins), insulin mimetics, such as a small-molecule insulin, e.g., L-783,281, insulin analogs (e.g., LYSPRO™ (Eli Lilly Co.), Lys^{B28}insulin, Pro^{B29}insulin, or Asp^{B28}insulin or those described in, for example, U.S. Pat. Nos. 5,149,777 and 5,514,646) or physiologically active fragments thereof, insulin-related peptides (C-peptide, GLP-1, IGF-1, or IGF-1/IGFBP-3 complex) or analogs or fragments thereof, ergoset, pramlintide, leptin, BAY-27-9955, T-1095, antagonists to insulin receptor tyrosine kinase inhibitor, antagonists to TNF-alpha function, a growth-hormone-releasing agent, amylin or antibodies to amylin, an insulin sensitizer, such as compounds of the glitazone family, including those described in U.S. Pat. No. 5,753,681, such as troglitazone, pioglitazone, englitazone, and related compounds, LINALOL™ alone or with Vitamin E (U.S. Pat. No. 6,187,333), and insulin secretion enhancers, such as nateglinide (AY-4166), calcium (2S)-2-benzyl-3-(cis-hexahydro-2-isoindolinylcarbonyl)propionate dihydrate (mitiglinide, KAD-1229), repaglinide, and sulfonylurea drugs, for example, acetohexamide, chlorpropamide, tolazamide, tolbutamide, glyclopyramide and its ammonium salt, glibenclamide, glibomuride, gliclazide, 1-butyl-3-metanilylurea, carbutamide, glipizide, gliquidone, glisoxepid, glybuthiazole, glibuzole, glyhexamide, glymidine, glypinamide, phenbutamide, tolcyclamide, glimepiride, etc., as well as biguanides (such as phenformin, metformin, buformin, etc.), and α-glucosidase inhibitors (such as acarbose, voglibose, miglitol, emiglitate, etc.), and such non-typical treatments as pancreatic transplant or autoimmune reagents.

As used herein, “insulin” refers to any and all substances having an insulin action, and exemplified by, for example, animal insulin extracted from bovine or porcine pancreas, semi-synthesized human insulin that is enyzmatically synthesized from insulin extracted from porcine pancreas, and human insulin synthesized by genetic engineering techniques typically using E. coli or yeasts, etc. Further, insulin can include insulin-zinc complex containing about 0.45 to 0.9 (w/w)% of zinc, protamine-insulin-zinc produced from zinc chloride, protamine sulfate and insulin, etc. Insulin may be in the form of its fragments or derivatives, e.g., INS-1. Insulin may also include insulin-like substances, such as L83281 and insulin agonists. While insulin is available in a variety of types, such as super immediate-acting, immediate-acting, bimodal-acting, intermediate-acting, long-acting, etc., these types can be appropriately selected according to the patient's condition.

As used herein, the term “transgene” refers to a nucleic acid sequence that is partly or entirely heterologous, i.e., foreign, to the transgenic animal into which it is introduced, or is homologous to an endogenous gene of the transgenic animal into which it is introduced, but which is designed to be inserted, or is inserted, into the animal's genome in such a way as to alter the genome of the cell into which it is inserted (e.g., it is inserted at a location that differs from that of the natural gene). A transgene can be operably linked to one or more transcriptional regulatory sequences and any other nucleic acid, such as introns, that may be necessary for optimal expression of a selected nucleic acid. The transgene herein encodes Dkk-5.

The “transgenic non-human animals” herein all include within a plurality of their cells the Dkk-5-encoding transgene, which alters the phenotype of the host cell with respect to glucose clearance in the blood.

“Isolated,” when used to describe the various polypeptides and protein fragments disclosed herein, means polypeptide or protein that has been identified and separated and/or recovered from a component of its natural environment. Contaminant components of its natural environment are materials that would typically interfere with diagnostic or therapeutic uses for the polypeptide or protein, and may include enzymes, hormones, and other proteinaceous or non-proteinaceous solutes. In preferred embodiments, the polypeptide will be purified (1) to a degree sufficient to obtain at least 15 residues of N-terminal or internal amino acid sequence by use of a spinning cup sequenator, or (2) to homogeneity by SDS-PAGE under non-reducing or reducing conditions using Coomassie blue or, preferably, silver stain. Isolated polypeptide includes polypeptide in situ within recombinant cells, since at least one component of the Dkk-1 natural environment will not be present. Ordinarily, however, isolated polypeptide will be prepared by at least one purification step.

Modes for Carrying Out the Invention

Based on the discovery herein of the actions of Dkk-5 on L6 muscle cells and other data, novel methods are disclosed for diagnosing and treating an insulin-resistant disorder using Dkk-5. Therefore, the present invention provides for methods useful in a number of in vitro and in vivo diagnostic and therapeutic situations.

Therapeutic Use

The Dkk-5 is administered to mammals by any suitable route, including a parenteral route of administration, such as, but not limited to, intravenous (IV), intramuscular (IM), subcutaneous (SC), and intraperitoneal (IP), as well as transdermal, buccal, sublingual, intrarectal, intranasal, and inhalant routes. IV, IM, SC, and IP administration may be by bolus or infusion, and in the case of SC, may also be by slow-release implantable device, including, but not limited to pumps, slow-release formulations, and mechanical devices. Preferably, administration is systemic and a decrease in insulin resistance is manifested in a drop in circulating levels of glucose and/or insulin in the patient.

One specifically preferred method for administration of Dkk-5 is by subcutaneous infusion, particularly using a metered infusion device, such as a pump. Such pump can be reusable or disposable, and implantable or externally mountable. Medication infusion pumps that are usefully employed for this purpose include, for example, the pumps disclosed in U.S. Pat Nos. 5,637,095; 5,569,186; and 5,527,307. The compositions can be administered continually from such devices, or intermittently.

Therapeutic formulations of Dkk-5 suitable for storage include mixtures of the protein having the desired degree of purity with pharmaceutically acceptable carriers, excipients, or stabilizers (Remington's Pharmaceutical Sciences 16th edition, Osol, A. Ed. (1980)), in the form of lyophilized formulations or aqueous solutions. Acceptable carriers, excipients, or stabilizers are non-toxic to recipients at the dosages and concentrations employed, and include buffers, such as phosphate, citrate, and other organic acids; anti-oxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens, such as methyl or propyl paraben; catechol; resorcinol; cyclohexanol; 3-pentanol; and m-cresol); low-molecular-weight (less than about 10 residues) polypeptides; proteins, such as serum albumin, gelatin, or immunoglobulins; hydrophilic polymers, such as polyvinylpyrrolidone; amino acids, such as glycine, glutamine, asparagine, histidine, arginine, or lysine; monosaccharides, disaccharides, and other carbohydrates including glucose, mannose, or dextrins; chelating agents, such as EDTA; sugars, such as sucrose, mannitol, trehalose, or sorbitol; salt-forming counter-ions, such as sodium; metal complexes (e.g., Zn-protein complexes); and/or non-ionic surfactants, such as TWEEN™, PLURONICS™ or polyethylene glycol (PEG). Preferred lyophilized Dkk-5 formulations are described in WO 97/04801. These compositions comprise Dkk-5 containing from about 0.1 to 90% by weight of the active Dkk-5, preferably in a soluble form, and more generally from about 10 to 30% by weight.

The active ingredients may also be entrapped in microcapsules prepared, for example, by coacervation techniques or by interfacial polymerization, for example, hydroxymethylcellulose or gelatin-microcapsules and poly(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences, supra.

The Dkk-5 disclosed herein may also be formulated as immunoliposomes. Liposomes containing the Dkk-5 are prepared by methods known in the art, such as described in Epstein et al., Proc. Natl. Acad. Sci. USA, 82: 3688 (1985); Hwang et al., Proc. Natl. Acad. Sci. USA, 77: 4030 (1980); U.S. Pat. Nos. 4,485,045 and 4,544,545; and WO97/38731 published Oct. 23, 1997. Liposomes with enhanced circulation time are disclosed in U.S. Pat. No. 5,013,556.

Particularly useful liposomes can be generated by the reverse-phase evaporation method with a lipid composition comprising phosphatidylcholine, cholesterol, and PEG-derivatized phosphatidylethanolamine (PEG-PE). Liposomes are extruded through filters of defined pore size to yield liposomes with the desired diameter.

Sustained-release preparations may be prepared. Suitable examples of sustained-release preparations include semipermeable matrices of solid hydrophobic polymers containing the Dkk-5, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and γ ethyl-L-glutamate, nondegradable ethylene-vinyl acetate, degradable lactic acid-glycolic acid copolymers, such as the LUPRON DEPOT™ (injectable microspheres composed of lactic acid-glycolic acid copolymer and leuprolide acetate), and poly-D-(−)-3-hydroxybutyric acid.

The Dkk-5 can be joined to a carrier protein to increase its serum half-life. The formulations to be used for in vivo administration must be sterile. This is readily accomplished by filtration through sterile filtration membranes.

The formulation herein may also contain more than one active compound as necessary for the particular indication being treated, preferably those with complementary activities that do not adversely affect each other. Also, such active compound can be administered separately to the mammal being treated. Such other drugs may be administered, by a route and in an amount commonly used therefor, contemporaneously or sequentially with the Dkk-5. When the Dkk-5 is used contemporaneously with one or more other drugs, a pharmaceutical unit dosage form containing such other drugs in addition to the Dkk-5 is preferred. Accordingly, the pharmaceutical compositions of the present invention include those that also contain one or more other active ingredients, in addition to the Dkk-5. Examples of insulin-resistance-treating agents or hypoglycemic agents that may be combined with the Dkk-5, either administered separately or in the same pharmaceutical compositions, include, but are not limited to:
a) insulin sensitizers including (i) PPAR-gamma agonists, such as the glitazones (e.g., including those described in U.S. Pat. No. 5,753,681, such as troglitazone (Noscal or Resiline), pioglitazone HCL, englitazone, MCC-555, BRL49653, ALRT 268, LGD 1069, chromic picolinate, DIAB II™ (V-411) or GLUCANIN™ and the like), and compounds disclosed in WO 97/27857, WO 97/28115, WO 97/28137, and WO 97/27847 and (ii) biguanides, such as metformin and phenformin;
(b) insulin (one or more different insulins), insulin mimetics, such as a small-molecule insulin, e.g., L-783,281, insulin analogs (e.g., LYSPRO™ (Eli Lilly Co.), Lys^{B28}insulin, Pro^{B29}insulin, or As 8insulin or those described in, for example, U.S. Pat. Nos. 5,149,777 and 5,514,646) or physiologically active fragments thereof, insulin-related peptides (C-peptide, GLP-1, IGF-1, or IGF-1/IGFBP-3 complex) or analogs or fragments thereof;
(c) sulfonylureas, such as acetohexamide, chlorpropamide, tolazamide, tolbutamide, glibenclaminde, glibomuride, gliclazide, glipizide, gliquidone and glymidine;
(d) alpha-glucosidase inhibitors (such as acarbose),
(e) cholesterol-lowering agents, such as (i) HMG-CoA reductase inhibitors (lovastatin, simvastatin and pravastatn, fluvastatin, atorvastatin, and other statins), (ii) sequestrants (cholestyramine, colestipol, and a dialkylaminoalkyl derivative of a cross-linked dextran), (iii) nicotinyl alcohol nicotinic acid or a salt thereof, (iv) proliferator-activator receptor-alpha agonists, such as fenofibric acid derivatives (gemfibrozil, clofibrat, fenofibrate, and benzafibrate), (v) inhibitors of cholesterol absorption, for example, beta-sitosterol and (acyl CoA:cholesterol acyltransferase) inhibitors, for example, melinamide, (vi) probucol, (vii) vitamin E, and (viii) thyromimetics;
(f) PPAR-delta agonists, such as those disclosed in WO 97/28149;
(g) anti-obesity compounds, such as fenfluramine, dexfenfluramine, phentermine, sibutramine, orlistat, and other beta₃ adrenergic receptor agonists;
(h) feeding behavior modifying agents, such as neuropeptide Y antagonists (e.g., neuropeptide Y5), for example, those disclosed in WO 97/19682, WO 97/20820, WO 97/20821, WO 97/20822 and WO 97120823;
(i) PPAR-alpha agonists, such as described in WO 97/36579;
(j) PPAR-gamma antagonists, such as described in WO 97/10813;
(k) serotonin reuptake inhibitors, such as fluoxetine and sertraline;
(l) one or more insulin sensitizers along with one or more of an orally ingested insulin, an injected insulin, a sulfonylurea, a biguanide or an alpha-glucosidase inhibitor as described in U.S. Pat. No. 6,291,495;
(m) autoimmune reagents;
(n) antagonists to insulin receptor tyrosine kinase inhibitor (U.S. Pat. Nos. 5,939,269 and 5,939,269);
(o) IGF-1/IGFBP-3 complex (U.S. Pat. No. 6,040,292);
(p) antagonists to TNF-alpha function (U.S. Pat. No. 6,015,558);
(q) growth hormone releasing agent (U.S. Pat. No. 5,939,387); and
(r) antibodies to amylin (U.S. Pat. No. 5,942,227).
   Other agents are specified in the definition above or are known to those skilled in the art.

Such additional molecules are suitably present or administered in combination in amounts that are effective for the purpose intended, typically less than what is used if they are administered alone without the Dkk-5. If they are formulated together, they may be formulated in the amounts determined according to, for example, the subject, the age and body weight of the subject, current clinical status, administration time, dosage form, administration method, etc. For instance, a concomitant drug is used preferably in a proportion of about 0.0001 to 10,000 weight parts relative to one weight part of the Dkk-5 herein.

The hypoglycemic agent is administered to the mammal by any suitable technique including parenterally, intranasally, orally, or by any other effective route. Most preferably, the administration is by injection (as of insulin) or by the oral route. For example, MICRONASE™ Tablets (glyburide) marketed by Upjohn in 1.25, 2.5, and 5 mg tablet concentrations are suitable for oral administration. The usual maintenance dose for Type II diabetics, placed on this therapy, is generally in the range of from about 1.25 to 20 mg per day, which may be given as a single dose or divided throughout the day as deemed appropriate (Physician's Desk Reference, 2563-2565 (1995)). Other examples of glyburide-based tablets available for prescription include GLYNASE™ brand drug (Upjohn) and DIABETA™ brand drug (Hoechst-Roussel). GLUCOTROL™ (Pratt) is the trademark for a glipizide (1-cyclohexyl-3-[p-[2-(5-methylpyrazine carboxamide)ethyl]phenyl]sulfonylurea) tablet available in both 5 and 10 mg strengths and is also prescribed to Type II diabetics who require hypoglycemic therapy following dietary control or in patients who have ceased to respond to other sulfonylureas (Physician's Desk Reference, 1902-1903 (1995)).

Use of the Dkk-5 in combination with insulin enables reduction of the dose of insulin as compared with the dose at the time of administration of insulin alone. Therefore, risk of blood vessel complication and hypoglycemia induction, both of which may be problems with large amounts of insulin administration, is low. For administration of insulin to an adult diabetic patient (body weight about 50 kg), for example, the dose per day is usually about 10 to 100 U (Units), preferably about 10 to 80 U, but this may be less as determined by the physician. For administration of insulin secretion enhancers to the same type of patient, for example, the dose per day is preferably about 0.1 to 1000 mg, more preferably about 1 to 100 mg. For administration of biguanides to the same type of patient, for example, the dose per day is preferably about 10 to 2500 mg, more preferably about 100 to 1000 mg. For administration of α-glucosidase inhibitors to the same type of patient, for example, the dose per day is preferably about 0.1 to 400 mg, more preferably about 0.6 to 300 mg. Administration of ergoset, pramlintide, leptin, BAY-27-9955, or T-1095 to such patients can be effected at a dose of preferably about 0.1 to 2500 mg, more preferably about 0.5 to 1000 mg. All of the above doses can be administered once to several times a day.

The Dkk-5 may also be administered together with a suitable non-drug treatment for an insulin-resistant disorder, such as a pancreatic transplant.

The dosages of Dkk-5 administered to an insulin-resistant mammal will be determined by the physician in the light of the relevant circumstances, including the condition of the mammal, and the chosen route of administration. The dosage ranges presented herein are not intended to limit the scope of the invention in any way. A “therapeutically effective” amount for purposes herein is determined by the above factors, but is generally about 0.01 to 100 mg/kg body weight/day. The preferred dose is about 0.1-50 mg/kg/day, more preferably about 0.1 to 25 mg/kg/day. More preferred still, when the Dkk-5 is administered daily, the intravenous or intramuscular dose for a human is about 0.3 to 10 mg/kg of body weight per day, more preferably, about 0.5 to 5 mg/kg. For subcutaneous administration, the dose is preferably greater than the therapeutically equivalent dose given intravenously or intramuscularly. Preferably, the daily subcutaneous dose for a human is about 0.3 to 20 mg/kg, more preferably about 0.5 to 5 mg/kg.

The invention contemplates a variety of dosing schedules. The invention encompasses continuous dosing schedules, in which Dkk-5 is administered on a regular (daily, weekly, or monthly, depending on the dose and dosage form) basis without substantial breaks. Preferred continuous dosing schedules include daily continuous infusion, where Dkk-5 is infused each day, and continuous bolus administration schedules, where Dkk-5 is administered at least once per day by bolus injection or inhalant or intranasal routes. The invention also encompasses discontinuous (e.g., intermittent and maintenance) dosing schedules. The exact parameters of such discontinuous administration schedules will vary according to the formulation, method of delivery, and the clinical needs of the mammal being treated. For example, if the Dkk-5 is administered by infusion, administration schedules may comprise a first period of administration followed by a second period in which Dkk-5 is not administered that is greater than, equal to, or less than the first period.

Where the administration is by bolus injection, especially bolus injection of a slow-release formulation, dosing schedules may also be continuous in that Dkk-5 is administered each day, or may be discontinuous, with first and second periods and so on as described above.

Continuous and discontinuous administration schedules by any method also include dosing schedules in which the dose is modulated throughout the first period, such that, for example, at the beginning of the first period, the dose is low and increased until the end of the first period, the dose is initially high and decreased during the first period, the dose is initially low, increased to a peak level, then reduced towards the end of the first period, and any combination thereof.

The effects of administration of Dkk-5 can be measured by a variety of assays known in the art. Most commonly, alleviation of the effects of diabetes will result in improved glycemic control (as measured by serial testing of blood glucose), reduction in the requirement for insulin to maintain good glycemic control, reduction in serum insulin levels, reduction in glycosylated hemoglobin, reduction in blood levels of advanced glycosylation end-products (AGE), reduced “dawn phenomenon”, reduced ketoacidosis, and improved lipid profile. Alternatively, administration of Dkk-5 can result in a stabilization of the symptoms of diabetes, as indicated by reduction of blood glucose levels, reduced insulin requirement, reduced serum insulin levels, reduced glycosylated hemoglobin and blood AGE, reduced vascular, renal, neural and retinal complications, reduced complications of pregnancy, and improved lipid profile.

The blood sugar lowering effect of the Dkk-5 can be evaluated by determining the concentration of glucose or Hb(hemoglobin)A_{1c} in venous blood plasma in the subject before and after administration, and then comparing the obtained concentration before administration and after administration. HbA_{1c} means glycosylated hemoglobin, and is gradually produced in response to blood glucose concentration. Therefore, HbA_{1c} is thought important as an index of blood sugar control that is not easily influenced by rapid blood sugar changes in diabetic patients.

The invention also provides kits for the treatment of an insulin-resistant disorder. The kits of the invention comprise one or more containers of Dkk-5 in a predetermined amount in combination with a set of instructions, generally written instructions, relating to the use and dosage of Dkk-5 for the treatment of an insulin-resistant disorder, preferably diabetes. The instructions included with the kit generally include information as to dosage, dosing schedule, and route of administration for the treatment of the insulin-resistant disorder. The containers of Dkk-5 may be unit doses, bulk packages (e.g., multi-dose packages), or sub-unit doses.

Dkk-5 may be packaged in any convenient, appropriate packaging. For example, if the Dkk-5 is a freeze-dried formulation, an ampoule or vial with a resilient stopper is normally used as the container, so that the drug may be easily reconstituted by injecting fluid through the resilient stopper. Ampoules with non-resilient, removable closures (e.g., sealed glass) or resilient stoppers are most conveniently used for injectable forms of Dkk-5. In this case, the instructions preferably specify placing the contents of the vial in a syringe for immediate injection. Also contemplated are packages for use in combination with a specific device, such as an inhaler, a nasal administration device (e.g., an atomizer), or an infusion device, such as a mini-pump.

The kit may also comprise a container comprising an insulin-resistance-treating agent in a predetermined amount.

Diagnostic Use

Many different assays and assay formats can be used to detect the amount of Dkk-5 in a sample relative to a control sample. These formats, in turn, are useful in the diagnostic assays of the present invention, which are used to detect the presence or onset of an insulin-resistant disorder in a mammal.

Any procedure known in the art for the measurement of soluble analytes can be used in the practice of the instant invention. Such procedures include, but are not limited to, competitive and non-competitive assay systems using techniques, such as radioimmunoassay, enzyme immunoassays (EIA), preferably ELISA, “sandwich” immunoassays, precipitin reactions, gel diffusion reactions, immunodiffusion assays, agglutination assays, complement-fixation assays, immunoradiometric assays, fluorescent immunoassays, protein A immunoassays, and immunoelectrophoresis assays. For examples of preferred immunoassay methods, see U.S. Pat. Nos. 4,845,026 and 5,006,459.

In one embodiment, one or more of anti-Dkk-5 antibodies are used to measure the amount of Dkk-5 in the sample. For diagnostic applications, if an anti-Dkk-5 antibody is used for detection, the antibody typically will be labeled with a detectable moiety. Preferably such antibody is used in an immunoassay. In one aspect of labeling, one or more of the anti-Dkk-5 antibodies used is labeled; in another aspect, a first antibody is unlabeled, and a labeled, second antibody is used to detect the Dkk-5 bound to the first antibody or is used to detect the first antibody.

Numerous labels are available, which can be generally grouped into the following categories:
(a) Radioisotopes, such as ³⁵S, ¹⁴C, ¹²⁵I, ³H, and ¹³¹I, are available. The antibody can be labeled with the radioisotope or radionuclide using the techniques described in Current Protocols in Immunology, Volumes 1 and 2, Coligen et al., Ed. (Wiley-Interscience: New York, 1991), for example, and radioactivity can be measured using scintillation counting.

(b) Fluorescent labels, such as rare-earth chelates (europium chelates) or fluorescein and its derivatives (such as fluorescein isothiocyanate), rhodamine and its derivatives, phycoerythrin, phycocyanin, allophycocyanin, o-phthaldehyde, fluorescamine, dansyl, lissamine, and Texas Red, are available. The fluorescent labels can be conjugated to the antibody using the techniques disclosed in Current Protocols in Immunology, supra, for example. Fluorescence can be quantified using a fluorimeter. The detecting antibody can also be detectably labeled using fluorescence-emitting metals, such as ¹⁵²Eu or others of the lanthanide series. These metals can be attached to the antibody using such metal-chelating groups as diethylenetriaminepentaacetic acid (DTPA) or ethylenediaminetetraacetic acid (EDTA).

(c) Various enzyme-substrate labels are available for an EIA, and U.S. Pat. No. 4,275,149 provides a review of some of these. The enzyme generally catalyzes a chemical alteration of the chromogenic substrate that can be measured using various techniques. For example, the enzyme may catalyze a color change in a substrate, which can be measured spectrophotometrically. Alternatively, the enzyme may alter the fluorescence, chemiluminescence, or bioluminescence of the substrate. Techniques for quantifying a change in fluorescence are described above. The chemiluminescent substrate becomes electronically excited by a chemical reaction and may then emit light that can be measured (using a chemiluminometer, for example) or donates energy to a fluorescent acceptor. Examples of enzymatic labels include luciferases (e.g., firefly luciferase and bacterial luciferase; U.S. Pat. No. 4,737,456), luciferin, aequorin, 2,3 dihydrophthalazinediones, malate dehydrogenase, urease, a peroxidase, such as horseradish peroxidase (HRPO), alkaline phosphatase, β-galactosidase, glucoamylase, lysozyme, saccharide oxidases (e.g., glucose oxidase, galactose oxidase, yeast alcohol dehydrogenase, alpha-glycerophosphate dehydrogenase, and glucose-β-phosphate dehydrogenase), staphylococcal nuclease, delta-V-steroid isomerase, triose phosphate isomerase, asparaginase, ribonuclease, urease, catalase, acetylcholinesterase, heterocyclic oxidases (such as uricase and xanthine oxidase), lactoperoxidase, microperoxidase, and the like. Techniques for conjugating enzymes to antibodies are described in O'Sullivan et al., Methods in Enzym., ed. Langone and Van Vunakis (Academic Press: New York) 73: 147-166 (1981).

Examples of enzyme-substrate combinations include:
(i) Horseradish peroxidase (HRPO) with hydrogen peroxidase as a substrate, wherein the hydrogen peroxidase oxidizes a dye precursor (e.g., orthophenylene diamine (OPD) or 3,3′,5,5′-tetramethyl benzidine hydrochloride (TMB));
(ii) alkaline phosphatase (AP) with para-nitrophenyl phosphate as chromogenic substrate; and
(iii) β-D-galactosidase (β-D-Gal) with a chromogenic substrate (e.g., p-nitrophenyl-β-D-galactosidase) or fluorogenic substrate 4-methylumbelliferyl-β-galactosidase.

Numerous other enzyme-substrate combinations are available to those skilled in the art. For a general review of these, see U.S. Pat. Nos. 4,275,149 and 4,318,980.

Sometimes, the label is indirectly conjugated with the antibody. The skilled artisan will be aware of various techniques for achieving this. For example, the antibody can be conjugated with biotin and any of the three broad categories of labels mentioned above can be conjugated with avidin, or vice versa. Biotin binds selectively to avidin, and thus, the label can be conjugated with the antibody in this indirect manner. Alternatively, to achieve indirect conjugation of the label with the antibody, the antibody is conjugated with a small hapten (e.g., digoxin) and one of the different types of labels mentioned above is conjugated with an anti-hapten antibody (e.g., anti-digoxin antibody). Thus, indirect conjugation of the label with the antibody can be achieved.

In another embodiment of the invention, the anti-Dkk-5 antibody need not be labeled, and the presence thereof can be detected using a labeled antibody that binds to the Dkk-5 antibody.

The antibodies of the present invention may be employed in any known assay method, such as competitive binding assays, direct and indirect sandwich assays, and immunoprecipitation assays. Zola, Monoclonal Antibodies: A Manual of Techniques, pp. 147-158 (CRC Press, Inc., 1987).

In the assays of the present invention, the antigen Dkk-5 or antibodies thereto are preferably bound to a solid phase support or carrier. By “solid phase support or carrier” is intended any support capable of binding an antigen or antibodies. Well known supports, or carriers, include glass, polystyrene, polypropylene, polyethylene, dextran, nylon, amyloses, natural and modified celluloses, polyacrylamides, agaroses, and magnetite. The nature of the carrier can be either soluble to some extent or insoluble for the purposes of the present invention. The support material may have virtually any possible structural configuration so long as the coupled molecule is capable of binding to an antigen or antibody. Thus, the support configuration may be spherical, as in a bead, or cylindrical, as in the inside surface of a test tube, or the external surface of a rod. Alternatively, the surface may be flat, such as a sheet, test strip, etc. Preferred supports include polystyrene beads. Those skilled in the art will know many other suitable carriers for binding antibody or antigen, or will be able to ascertain the same by use of routine experimentation.

In a preferred embodiment, an antibody-antigen-antibody sandwich immunoassay is performed, i.e., antigen is detected or measured by a method comprising binding of a first antibody to the antigen, and binding of a second antibody to the antigen, and detecting or measuring antigen immunospecifically bound by both the first and second antibody. In a specific embodiment, the first and second antibodies are monoclonal antibodies. In this embodiment, if the antigen does not contain repetitive epitopes recognized by the monoclonal antibody, the second monoclonal antibody must bind to a site different from that of the first antibody (as reflected, e.g., by the lack of competitive inhibition between the two antibodies for binding to the antigen). In another specific embodiment, the first or second antibody is a polyclonal antibody. In yet another specific embodiment, both the first and second antibodies are polyclonal antibodies.

In a preferred embodiment, a “forward” sandwich enzyme immunoassay is used, as described schematically below. An antibody (capture antibody, Ab1) directed against the Dkk-5 is attached to a solid phase matrix, preferably a microplate. The sample is brought in contact with the Ab1-coated matrix such that any Dkk-5 in the sample to which Ab1 is specific binds to the solid-phase Ab1. Unbound sample components are removed by washing. An enzyme-conjugated second antibody (detection antibody, Ab2) directed against a second epitope of the antigen binds to the antigen captured by Ab1 and completes the sandwich. After removal of unbound Ab2 by washing, a chromogenic substrate for the enzyme is added, and a colored product is formed in proportion to the amount of enzyme present in the sandwich, which reflects the amount of antigen in the sample. The reaction is terminated by addition of stop solution. The color is measured as absorbance at an appropriate wavelength using a spectrophotometer. A standard curve is prepared from known concentrations of the antigen, from which unknown sample values can be determined.

Other types of “sandwich” assays are the so-called “simultaneous” and “reverse” assays. A simultaneous assay involves a single incubation step as the antibody bound to the solid support and labeled antibody are both added to the sample being tested at the same time. After the incubation is completed, the solid support is washed to remove the residue of fluid sample and uncomplexed labeled antibody. The presence of labeled antibody associated with the solid support is then determined as it would be in a conventional “forward” sandwich assay.

In the “reverse” assay, stepwise addition first of a solution of labeled antibody to the fluid sample followed by the addition of unlabeled antibody bound to a solid support after a suitable incubation period is utilized. After a second incubation, the solid phase is washed in conventional fashion to free it of the residue of the sample being tested and the solution of unreacted labeled antibody. The amount of labeled antibody associated with a solid support is then determined as in the “simultaneous” and “forward” assays.

Kits comprising one or more containers or vials containing components for carrying out the assays of the present invention are also within the scope of the invention. Such kit is a packaged combination of reagents in predetermined amounts with instructions for performing the diagnostic assay. For instance, such a kit can comprise an antibody or antibodies, preferably a pair of antibodies to the Dkk-5 antigen that preferably do not compete for the same binding site on the antigen. In a specific embodiment, Dkk-5 may be pre-adsorbed to the solid phase matrix. The kit preferably contains the other necessary washing reagents well known in the art. For EIA, the kit contains the chromogenic substrate as well as a reagent for stopping the enzymatic reaction when color development has occurred. The substrate included in the kit is one appropriate for the enzyme conjugated to one of the antibody preparations. These are well known in the art, and some are exemplified below. The kit can optionally also comprise a Dkk-5 standard; ie., an amount of purified Dkk-5 corresponding to a normal amount of Dkk-5 in a standard sample.

Where the antibody is labeled with an enzyme, the kit will include substrates and cofactors required by the enzyme (e.g., a substrate precursor that provides the detectable chromophore or fluorophore). In addition, other additives may be included, such as stabilizers, buffers (e.g., a block buffer or lysis buffer), and the like. The relative amounts of the various reagents may be varied widely to provide for concentrations in solution of the reagents that substantially optimize the sensitivity of the assay. Particularly, the reagents may be provided as dry powders, usually lyophilized, including excipients that on dissolution will provide a reagent solution having the appropriate concentration.

In one specific embodiment, a diagnostic kit for detecting the presence or onset of an insulin-resistant disorder comprises: (1) a container comprising an antibody that binds Dkk-5; (2) a container comprising a standard sample containing Dkk-5; and (3) instructions for using the antibody and standard sample to detect the disorder, wherein either the antibody that binds Dkk-5 is detectably labeled or the kit further comprises another container comprising a second antibody that is detectably labeled and binds to the Dkk-5 or to the antibody that binds Dkk-5. Preferably, the antibody that binds Dkk-5 is a monoclonal antibody.

In another specific embodiment, a kit of the invention comprises in one or more containers: (1) a solid phase carrier, such as a microtiter plate coated with a first antibody; (2) a detectably labeled second antibody; and (3) a standard sample of the Dkk-5 molecule recognized by the first and second antibodies, as well as appropriate instructions.

Screening Using Transgenic Animals

Transgenic non-human animals overexpressing dkk-5 cDNA in muscle cells can be used to screen candidate drugs (proteins, peptides, polypeptides, small molecules, etc.) for efficacy in increasing glucose clearance from the blood, indicating a treatment for an insulin-resistant disorder.

In one embodiment, the transgenic animals are produced by introducing the dkk-5 transgene into the germline of the non-human animal. Embryonal target cells at various developmental stages can be used to introduce transgenes. Different methods are used depending on the stage of development of the embryonal target cell. The specific line(s) of any animal used to practice this invention are selected for general good health, good embryo yields, good pronuclear visibility in the embryo, and good reproductive fitness. In addition, the haplotype is a significant factor. For example, when transgenic mice are to be produced, strains such as C57BL/6 or FVB lines are often used. The line(s) used to practice this invention may themselves be transgenics, and/or may be knockouts (i.e., obtained from animals that have one or more genes partially or completely suppressed).

The transgene construct may be introduced into a single-stage embryo. The zygote is the best target for micro-injection. The use of zygotes as a target for gene transfer has a major advantage in that in most cases the injected DNA will be incorporated into the host gene before the first cleavage (Brinster et al., Proc. Natl. Acad. Sci. USA, 82: 44384442 (1985)). As a consequence, all cells of the transgenic animal will carry the incorporated transgene. This will in general also be reflected in the efficient transmission of the transgene to offspring of the founder, since 50% of the germ cells will harbor the transgene.

Normally, fertilized embryos are incubated in suitable media until the pronuclei appear. At about this time, the nucleotide sequence comprising the transgene is introduced into the female or male pronucleus. In some species, such as mice, the male pronucleus is preferred. The exogenous genetic material may be added to the male DNA complement of the zygote prior to its being processed by the ovum nucleus or the zygote female pronucleus.

Thus, the exogenous genetic material may be added to the male complement of DNA or any other complement of DNA prior to its being affected by the female pronucleus, which is when the male and female pronuclei are well separated and both are located close to the cell membrane. Alternatively, the exogenous genetic material could be added to the nucleus of the sperm after it has been induced to undergo decondensation. Sperm containing the exogenous genetic material can then be added to the ovum or the decondensed sperm could be added to the ovum with the transgene constructs being added as soon as possible thereafter.

Any technique that allows for the addition of the exogenous genetic material into nucleic genetic material can be utilized so long as it is not destructive to the cell, nuclear membrane, or other existing cellular or genetic structures. Introduction of the transgene nucleotide sequence into the embryo may be accomplished by any means known in the art, such as, for example, microinjection, electroporation, or lipofection. The exogenous genetic material is preferentially inserted into the nucleic genetic material by microinjection. Microinjection of cells and cellular structures is known and is used in the art. In the mouse, the male pronucleus reaches the size of approximately 20 micrometers in diameter, which allows reproducible injection of 1-2 μl of DNA solution. Following introduction of the transgene nucleotide sequence into the embryo, the embryo may be incubated in vitro for varying amounts of time, or reimplanted into the surrogate host, or both. In vitro incubation to maturity is within the scope of this invention. One common method is to incubate the embryos in vitro for about 1-7 days, depending on the species, and then reimplant them into the surrogate host.

The number of copies of the transgene constructs that are added to the zygote depends on the total amount of exogenous genetic material added and will be the amount that enables the genetic transformation to occur. Theoretically only one copy is required; however, generally numerous copies are utilized, for example, 1,000-20,000 copies of the transgene construct, to ensure that one copy is functional. As regards the present invention, there may be an advantage to having more than one functioning copy of the inserted exogenous DNA sequence to enhance the phenotypic expression thereof.

Transgenic offspring of the surrogate host may be screened for the presence and/or expression of the transgene by any suitable method. Screening is often accomplished by Southern blot or Northern blot analysis, using a probe that is complementary to at least a portion of the transgene. Western blot analysis using an antibody against the Dkk-5 encoded by the transgene may be employed as an alternative or additional method for screening for the presence of the transgene product. Typically, DNA is prepared from tail tissue and analyzed by Southern analysis or PCR for the transgene. Alternatively, the tissues or cells believed to express the transgene at the highest levels are tested for the presence and expression of the transgene using Southern analysis or PCR, although any tissues or cell types may be used for this analysis.

Alternative or additional methods for evaluating the presence of the transgene include, without limitation, suitable biochemical assays, such as enzyme and/or immunological assays, histological stains for particular marker or enzyme activities, flow cytometric analysis, and the like. Analysis of the blood may also be useful to detect the presence of the transgene product in the blood, as well as to evaluate the effect of the transgene on the levels of blood constituents, such as glucose.

Progeny of the transgenic animals may be obtained by mating the transgenic animal with a suitable partner, or by in vitro fertilization of eggs and/or sperm obtained from the transgenic animal. Where mating with a partner is to be performed, the partner may or may not be transgenic and/or a knockout; where it is transgenic, it may contain the same or a different transgene, or both. Alternatively, the partner may be a parental line. Where in vitro fertilization is used, the fertilized embryo may be implanted into a surrogate host or incubated in vitro, or both. Using either method, the progeny may be evaluated for the presence of the transgene using methods described above, or other appropriate methods.

The transgene animals produced in accordance with this invention will include exogenous genetic material, i.e., a DNA sequence that results in the production of Dkk-5. The sequence will be attached operably to a a transcriptional control element, e.g., promoter, which preferably allows the expression of the transgene production in a specific type of cell. The most preferred such control element herein is a muscle-specific promoter that enables overexpression of the dkk-5 cDNA in muscle tissue. An example of such promoter is the myosin light-chain promoter (Shani, Nature, 314: 283-6 (1985)), or that driving smoothelin A or B expression, or similar such promoters, as described, for example, in WO 01/18048 published 15 Mar. 2001.

Retroviral infection can also be used to introduce the transgene into a non-human animal. The developing non-human embryo can be cultured in vitro to the blastocyst stage. During this time, the blastomeres can be targets for retroviral infection (Jaenich, Proc. Natl. Acad. Sci. USA, 73: 1260-1264 (1976)). Efficient infection of the blastomeres is obtained by enzymatic treatment to remove the zona pellucida (Manipulating the Mouse Embryo, Hogan, ed. (Cold Spring Harbor Laboratory Press, Cold Spring Harbor, N.Y., 1986)). The viral vector system used to introduce the transgene is typically a replication-defective retrovirus carrying the transgene (Jahner et al., Proc. Natl. Acad. Sci. USA, 82: 6972-6931 (1985); Van der Putten et al., Proc. Natl. Acad. Sci. USA, 82: 6148-6152 (1985)). Transfection is easily and efficiently obtained by culturing the blastomeres on a monolayer of virus-producing cells (Van der Putten et al., supra; Stewart et al., EMBO J., 6: 383-388 (1987)). Alternatively, infection can be performed at a later stage. Virus or virus-producing cells can be injected into the blastocoele (Jahner et al., Nature, 298: 623-628 (1982)). Most of the founders will be mosaic for the transgene, since incorporation occurs only in a subset of the cells that formed the transgenic non-human animal. Further, the founder may contain various retroviral insertions of the transgene at different positions in the genome that generally will segregate in the offspring. In addition, it is also possible to introduce transgenes into the germ line by intrauterine retroviral infection of the midgestation embryo (Jahner et al. (1982), supra).

A third type of target cell for transgene introduction is the embryonal stem cell (ES). ES cells are obtained from pre-implantation embryos cultured in vitro and fused with embryos (Evans et al., Nature, 292: 154-156 (1981); Bradley et al., Nature, 309: 255-258 (1984); Gossler et al., Proc. Natl. Acad. Sci. USA 83: 9065-9069 (1986)); Robertson et al., Nature, 322: 445-448 (1986)). Transgenes can be efficiently introduced into the ES cells by DNA transfection or by retrovirus-mediated transduction. Such transformed ES cells can thereafter be combined with blastocysts from a non-human animal. The ES cells thereafter colonize the embryo and contribute to the germ line of the resulting chimeric animal. For a review, see Jaenisch, Science, 240: 1468-1474 (1988).

Candidate drugs are screened for their ability to treat an insulin-resistant disorder by providing them to such animals (by, for example, inhalation, ingestion, injection, implantation, etc.) in an amount appropriate for glucose clearance or uptake potential to be measured. Increased glucose clearance or uptake would be indicative of the drug's ability to treat diabetes and other insulin-resistance disorders.

Gene Therapy with Dkk-5

Dkk-5 can be used in gene therapy for treating diabetes. Various approaches can be taken, such as cutaneous gene therapy or retroviral vector gene therapy to correct leptin deficiency, which produces a phenotype of reduced adipose tissue and insulin-resistance as well as congenital obesity and diabetes in humans (Larcher et al., FASEB J., 15: 1529-1538 (2001)). Another method for restoring insulin-sensitivity through gene therapy is to use adenovirus-mediated gene therapy as described in Ueki et al., J. Clin Invest., 105: 1437-1445 (2000). A further method is to use gene therapy to counteract diabetic hyperglycemia by engineering skeletal muscle to express Dkk-5-encoding DNA, as described by Otaegui et al., Human Gene Therapy, 11: 1543-1552 (2000).

The following Examples are set forth to assist in understanding the invention and should not, of course, be construed as specifically limiting the invention described and claimed herein. Such variations of the invention that would be within the purview of those in the art, including the substitution of all equivalents now known or later developed, are to be considered to fall within the scope of the invention as hereinafter claimed. The disclosures of all citations herein are incorporated by reference.

### EXAMPLE 1

### Effects of Dkk-5

Materials and Methods

L6 Cell Culture

L6 myoblasts were proliferated in growth medium, composed of MEM alpha (Gibco-BRL) with 10% fetal calf serum. Before confluence was reached the cells were dispersed with trypsin and seeded again in fresh growth medium. Myoblast fusion was induced by changing the medium to differentiation medium at confluence (MEM alpha with 2% fetal calf serum). Cells were grown in this medium for 3-9 days and for treatments longer than 28 hours, Dkk-5 was added to this medium. Treatments shorter than 28 hrs were performed in MEM alpha with 0.5% fetal bovine serum (FBS).

Expression of Recombinant Dkk-5

The human homolog of Dkk-5 (hDkk-5) (see SEQ ID NO:5 of FIG. 2 herein) was expressed in baculovirus-infected insect cells as a C-terminal 8×His tag fusion and purified by nickel affinity column chromatography (WO 01/40465 and WO 01/16319). The identity of purified protein was verified by N-terminal sequence analysis. The purified protein was less than 0.3 EU/ml endotoxin levels.

DOG Uptake

Control cells and cells treated with Dkk-5 were incubated in Krebs-Ringer phosphate-HEPES buffer (KRHB) (130 mM NaCl, 5 mM KCl, 1.3 mM CaCl₂, 1.3 mM MgSO₄, 10 mM Na2HPO₄, and 2s mM HEPES, pH 7.4) containing 0.5 μCi of 2-deoxy [¹⁴C] glucose in the presence or absence of 0.5 μM insulin for 20 min at 37° C. The cells were washed twice with KRHB and lysed in 100 mM NaOH, and the amount of intracellular 2-deoxy[¹⁴C] glucose in the cell lysates was measured by liquid scintillation (LSC).

Glycogen Synthesis

Glycogen synthesis was determined as [¹⁴C] glucose incorporation into glycogen. Control L6 cells and cells treated with Dkk-5 were incubated for 2 hours in serum-free MEM alpha containing [U-¹⁴C] glucose (5 mM glucose; 1.25 μCi/ml) with or without 0.5 μM insulin. The experiment was terminated by removing the medium and rapidly washing the cells three times with ice-cold PBS, and lysing them with 20% (w/v) KOH, which was neutralized after 1 hour by the addition of 1 M HCl. The lysates were boiled for 5 min and clarified by centrifugation, and the cellular glycogen in the supernatant was precipitated with isopropanol at 0° C. for 2 hours using 1 mg/ml cold glycogen as a carrier. The precipitated glycogen was separated by centrifugation, washed with 70% ethanol, and redissolved in water, and the incorporation of [¹⁴C] glucose into the glycogen was determined by LSC.

Glucose Incorporation into Lipids

Control and treated 3T3 L1 adipocytes were incubated with D-[U-14C]glucose (0.2 μCi/ml) in serum-free MEM alpha, for 2 hours at 37° C. in the presence or absence of 0.5 μM insulin. The cells were washed twice with ice-cold PBS and lysed in 100 mM NaOH. The lysates were neutralized with 100 mM hydrochloric acid. The cellular lipids in the lysates were extracted into n-heptane, and the incorporation of [¹⁴C] glucose into the extracted lipid was measured by liquid scintillation counter (LSC).

Real-Time Quantitative PCR

RTQ-PCR was performed using an ABI PRISM 7700™ Sequence Detection System instrument and software (PE Applied Biosystems, Inc., Foster City, Calif.) as described by Gibson et al., Genome Res., 6: 995-1001 (1996) and Heid et al., Genome Res., 6: 986-994 (1996).

Analysis

Unless otherwise noted, all data are presented as the means plus and minus the standard deviations. Comparisons between control and treated cells and between transgenic and wild-type mice were made using an unpaired student's t test.

Culture of 3T3/L1 Adipocytes

3T3/L1 fibroblasts were grown to confluence and differentiated to adipocytes (Rubin et al., J. Biol. Chem., 253: 7570-7578 (1978)). Differentiated cells were treated with Dkk-5 at 72 hours after the induction of differentiation.

Animals

All protocols would be approved by an Institutional Use and Care Committee. Unless otherwise noted, mice are maintained on standard lab chow in a temperature- and humidity-controlled environment. A 12-hour (6.00 pm/6.00 am) light cycle is used.

Transgenic Mice

The human dkk-5 cDNA was ligated 3′ to the pRK splice donor/acceptor site that is preceded by the myosin light-chain promoter (Shani, Nature 314: 283-6 (1985)). The dkk-5 cDNA was followed by the splice donor/acceptor sites present between the fourth and fifth exons of the human growth hormone gene (Stewart et al., Endocrinology, 130: 405-414 (1992)). The entire expression fragment was purified free from contaminating vector sequences and injected into one-cell mouse eggs derived from FVB X FVB matings. Transgenic mice were identified by PCR analysis of DNA extracted from tail biopsies.

Results

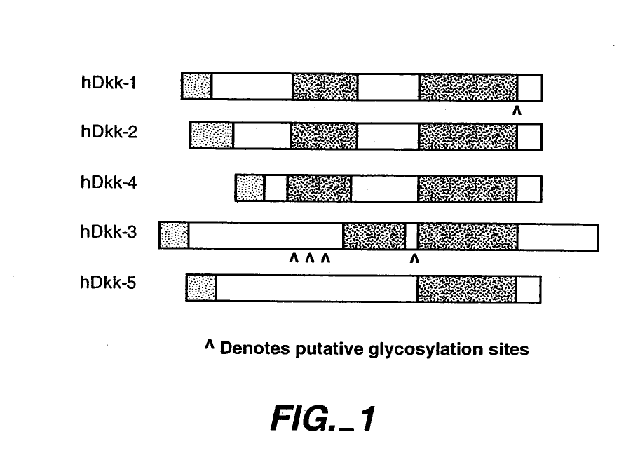

Dkk-5 is a secreted protein that is highly related to the dickkopf family of proteins. See FIGS. 1 and 2. Using radiation hybrid mapping, the gene for Dkk-5 was localized to chromosome 1 between DIS434 (32.2 cM) and DIS2843 (48.8 cM) by the present inventors. This location is confirmed by the data from other sequencing efforts as determined by BLAST analysis of the public sequence databases (see below).
HS330O12 Homo sapiens chromosome 1 clone RP3-330012 map p36.11-36.23,
***SEQUENCING IN PROGRESS***, in ordered pieces. 119969 bp
   DNA, RTG 28 Jun. 2001
ACCESSION AL031731
VERSION AL031731.36 GI:14575526
SOURCE human.
ORGANISM Homo sapiens
REFERENCE 1 (bases 1 to 119969)
AUTHORS Martin, S.
TITLE Direct Submission
JOURNAL Submitted (26 Jun. 2001) Sanger Centre, Hinxton, Cambridgeshire, CB10 1SA, UK.
COMMENT On Jun. 28, 2001 this sequence version replaced gi: 14422201.

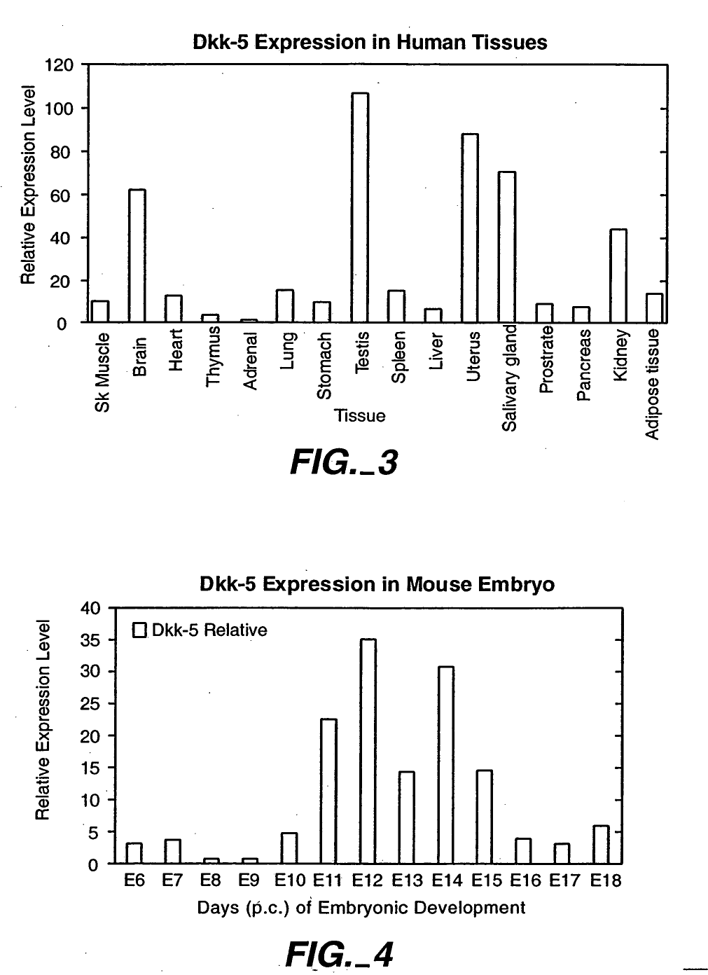

Dkk-5 was found to be widely expressed in adult human tissues, as shown in FIG. 3. This was determined by real-time quantitative PCR as described above.

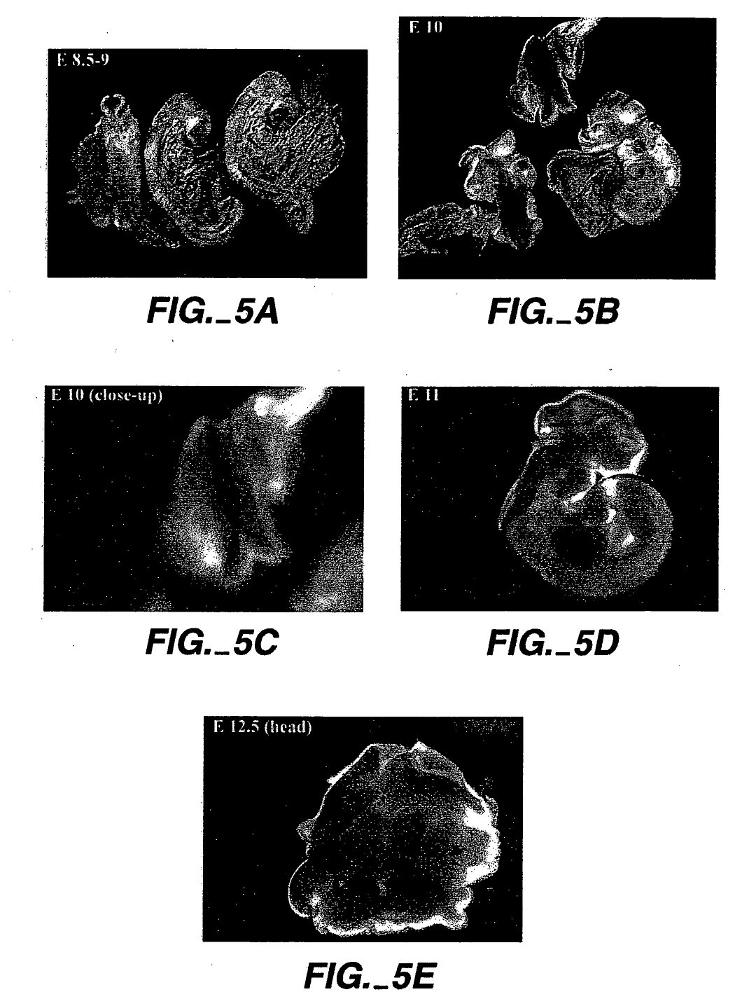

Dkk-5 was differentially expressed during mouse embryonic development. Real-time quantitative RT-PCR analysis of mouse embryos revealed that Dkk-5 expression begins at day 10 p.c. and continues until day 16 p.c. with the peak at day 12 p.c. See FIG. 4. In situ hybridization analysis of whole embryos showed that this expression is at the midbrain-hindbrain junction and along the roof plate, a region important in specification of mesoderm development. See FIG. 5.

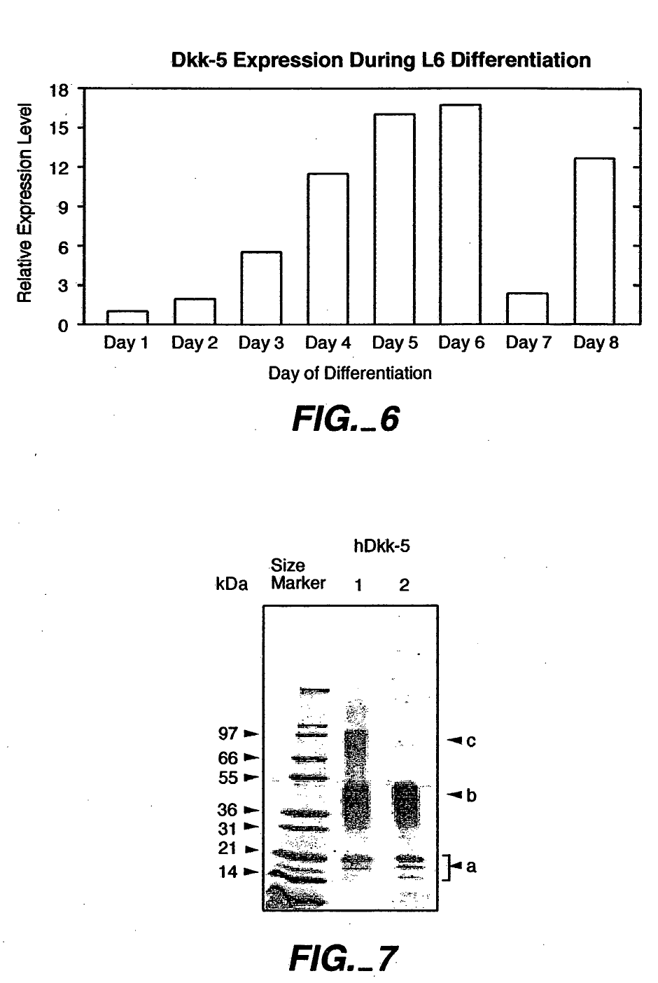

The results show that Dkk-5 expression was regulated during differentiation of L6 muscle cells. The levels of the transcript, as measured by real-time quantitative RT-PCR, started increasing at day 3 of differentiation and began to drop by day 7 of differentiation. See FIG. 6, which shows the relative expression level of Dkk-5 during L6 cell differentiation from day 1 to day 8. This expression pattern corresponds to the time period during which L6 cells are responsive to Dkk-5 and also to the period during which Dkk-5 binding to L6 cells is detectable.

When expressed in baculovirus-infected insect cells, the full-length Dkk-5 protein was clipped internally to give three cleavage products ranging from 16-kDa to 20-kDa in size. In the gel shown in FIG. 7, band “b” corresponds to the full-length protein. The N-terminal sequence of the full-length protein including signal sequence is MAGPAIHTAPML (SEQ ID NO:6). The mature protein starts at GALAPGTP (SEQ ID NO:7), so that the signal peptide cleavage site is between the alanine at position 24 and the glycine at position 25 in SEQ ID NO:5. The bands grouped as “a” correspond to the internally clipped proteins, all with N-terminal sequence MALFDWTDYEDLK (SEQ ID NO:8). The protein forms dimers (band c, lane 1 of FIG. 7), which get converted to the monomeric form under reducing conditions. The 16-kDa clipped protein, after largely purified (to about 90% purity) from the preparation of recombinantly produced full-length Dkk-5 by anion-exchange chromatography using a MONO-Q™-brand column, enhanced basal and insulin-stimulated glucose uptake in muscle cells. The Dkk-5 referred to in the experiments below was a preparation characterized as a mixture of full-length and internally clipped protein, containing approximately 5% clipped protein.

The clipped protein fragment may be purified from the full-length recombinant protein and any other undesired proteins by means of any classic protein chemistry technique, not limited to ion-exchange chromatography. In addition, large amounts of the full-length Dkk-5 protein may be expressed with limited proteolysis to obtain mostly clipped material; the Arg-Arg site in the molecule may also be clipped and the resulting desired cleavage product purified by size-exclusion or other conventional protein purification techniques well known to those skilled in the art.

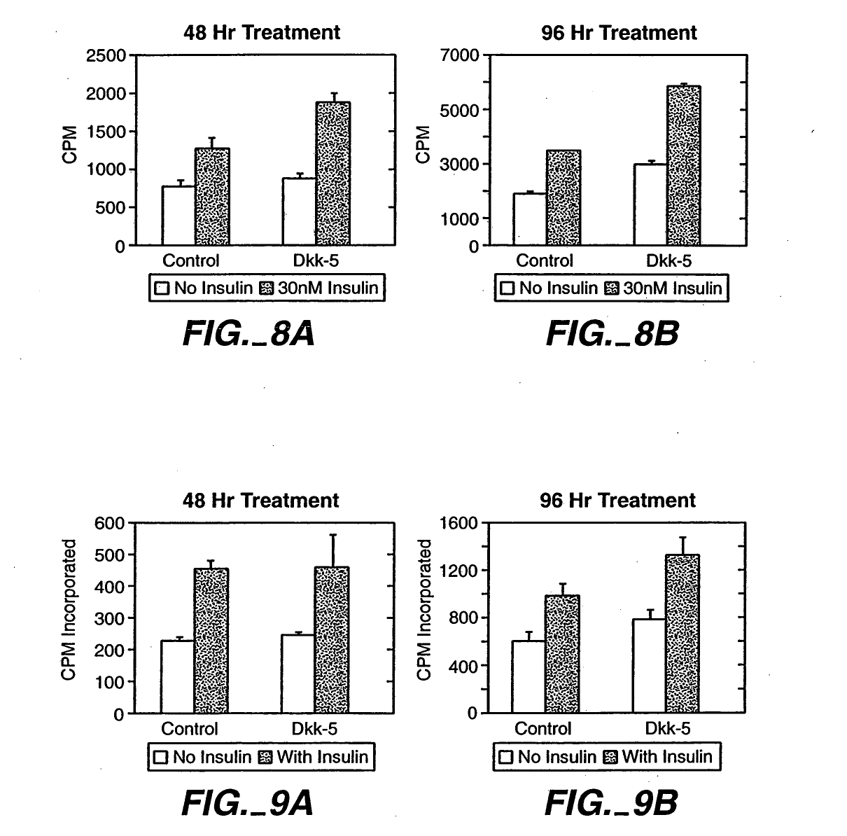

Treatment of L6 muscle cells with Dkk-5 resulted in an increased glucose (2-DOG) uptake. See FIG. 8. The effect of Dkk-5 can be seen within 48 hours (FIG. 8A) and depends on the differentiation state of the cells. The effects of Dkk-5 treatment on the increase in insulin-dependent glucose uptake are more significant at 96 hours (p=0.001) (FIG. 8B), although the effect is seen even at 48 hours (p=0.05).

Treatment of L6 muscle cells with Dkk-5 resulted in an increased incorporation of glucose into glycogen. See FIG. 9. As shown in FIG. 9A, the effects of Dkk-5 can be seen in 48 hours (p=0.003), and, without being limited to any one theory, this action may be mediated through regulation of activity of Akt and/or GSK-3β, both of which are intermediates in the Wnt and insulin signaling pathways.

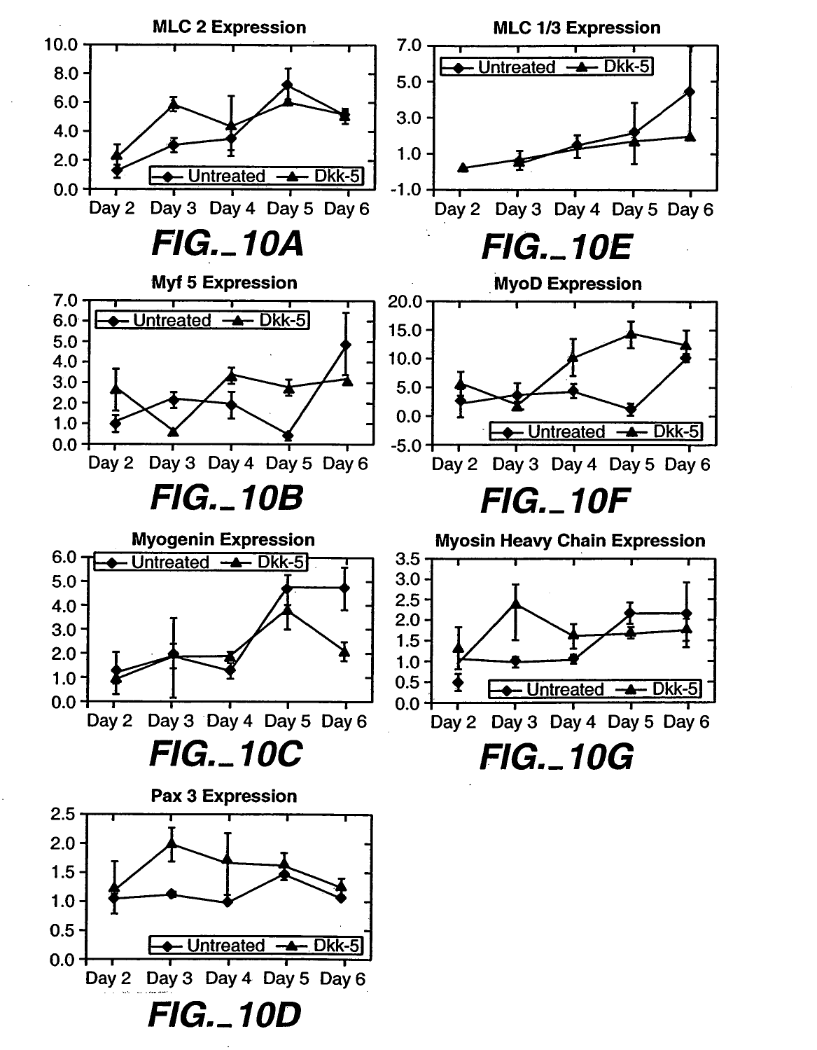

Dkk-5 affected myogenesis in L6 cells. Since the effects of Dkk-5 were observed following long-term treatment, it is possible, without being limited to any one theory, that the protein acts by affecting the differentiation of L6 cells. RT-PCR analysis using TAQMAN™ PCR was carried out to determine the expression levels of genes involved in myogenesis, such as myosin heavy chain (MHC), myosin light chain (MLC), myogenin, Pax3, Myf5, and MyoD in L6 cells treated with Dkk-5. FIGS. 10A-G-show that Dkk-5 treatment resulted in altered expression of myogenin and MyoD between days 4 and 6 of differentiation, and of MLC2, Myf5, and Pax 3 between days 2 and 4 of differentiation.

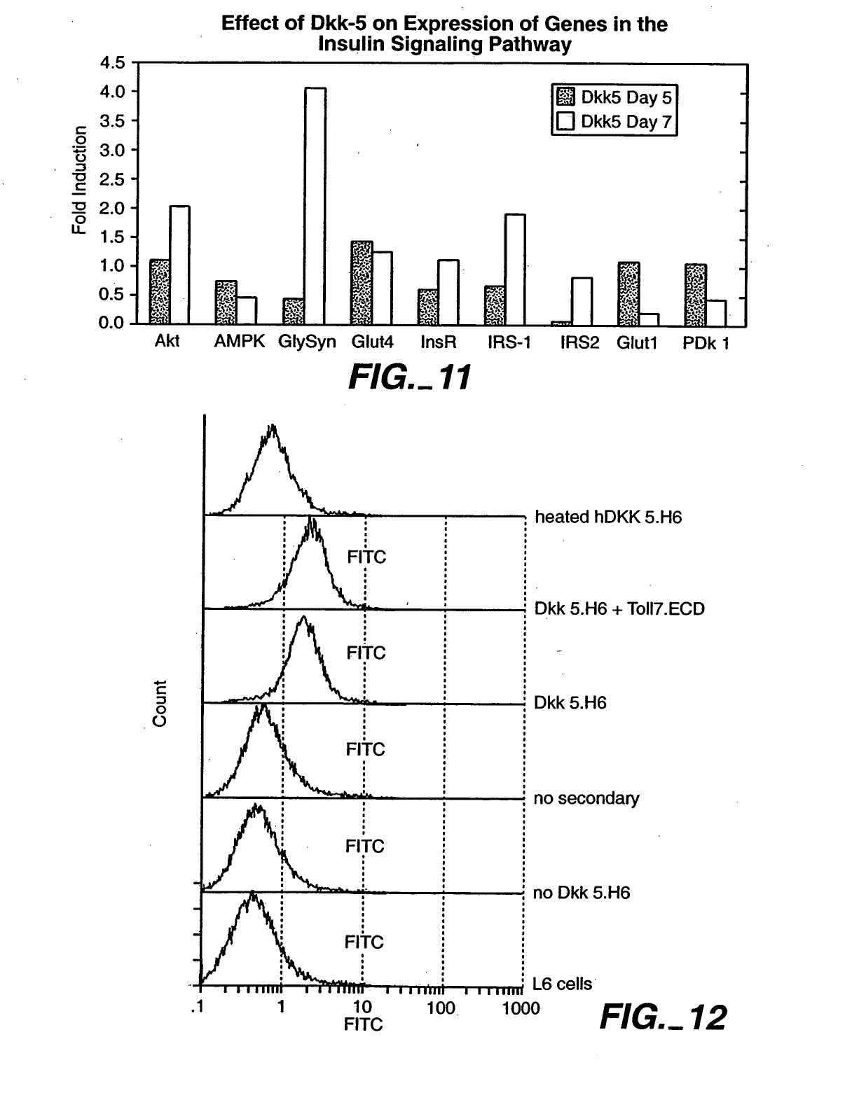

Dkk-5 regulated the expression of genes in the insulin-signaling pathway in muscle cells. RT-PCR analysis (TAQMAN™) was carried out to determine whether Dkk-5 affected the expression levels of genes involved in glucose metabolism. As shown in FIG. 11, Dkk-5 treatment increased the expression of Akt (2-fold), glycogen synthase (4-fold), and IRS-1 (2-fold) after 96 hours and decreased the expression of IRS-2 (0.2-fold after 48 hours treatment) and Glut-1 and PDK-1 (after 96 hours).

Using FACS analysis with polyclonal antibodies against Dkk-5 and monoclonal antibodies against the His 8 epitope tag, it was demonstrated that Dkk-5 binds L6 cells from day 2 through day 5 of differentiation, but this binding is decreased/lost by day 6. Dkk-S binding to L6 can be abolished by denaturing the protein, can be competed out by using excess Fc-Tagged Dkk-5, and is not affected by excess of unrelated His-tagged protein, suggesting that it is a specific interaction. See FIG. 12. Hence, Dkk-5 has a specific receptor on the surface of muscle cells. The related protein Dkk-1 binds LRP6, and, without being limited to any one theory, it is likely that Dkk-5 may also act through this receptor. These receptors were found by the instant inventors to be expressed on the surface of L6 cells and found by others to be expressed in normal muscle in mice and humans (Hey et al., Gene, 216: 103-111 (1998); Brown et al., Biochem. Biophys. Res. Commun., 248: 879-888 (1998)).

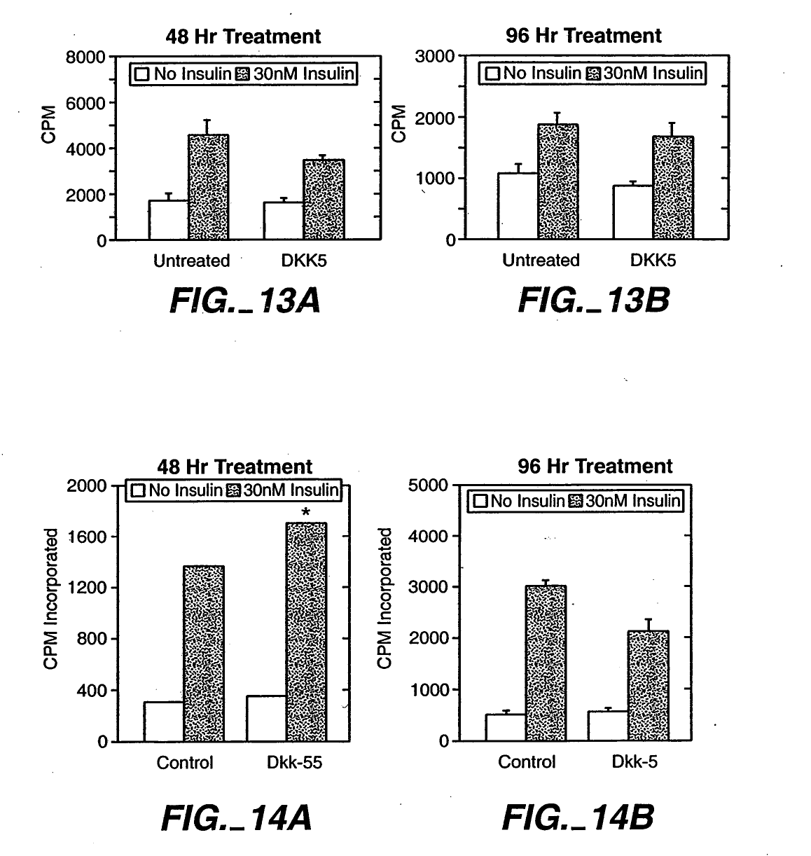

Dkk-5 treatment decreased basal and insulin-stimulated glucose uptake in adipocytes. Specifically, Dkk-5-treated 3T3 L1 cells showed an increase in levels of basal and insulin-stimulated glucose uptake (FIGS. 13A and 13B) as well as an increased incorporation of glucose into lipids following insulin stimulation (FIGS. 14A and 14B). The increase in insulin-dependent glucose uptake-seen at 48-hour treatment was more pronounced following 96-hour treatment, and a similar observation was seen with the insulin-dependent incorporation of glucose into lipid.

The effects of Dkk-5 in vivo were determined by analyzing the glucose metabolism of transgenic mice expressing the Dkk-5 cDNA under the control of a muscle-specific promoter (Shani, supra). Preliminary results showed that these particular transgenic animals did not have any altered glucose metabolism. Without being limited to any one theory, this result could be due to low expression, improper or lack of cleavage of the protein in these animals, or lack of secretion of the protein from muscle cells into neighboring cells, thereby accounting for the absence of any visible effects on glucose metabolism. Using a different promoter or other expression system such as a different splice donor/acceptor site at either end of the dkk-5 DNA is expected to lead to higher expression. In addition, expression of cDNA encoding only an active cleavage product of Dkk-5, such as the 16-kDa internal cleavage product, using proper start codons and other elements in the expression construct as would be apparent to the skilled practitioner, would enable determination of its effects on glucose metabolism in these transgenic animals.

Summary and Discussion

Dkk-5 had distinct effects on glucose uptake in muscle cells and in adipocytes. Dkk-5-treated muscle cells were more sensitive to insulin treatment. In muscle cells, Dkk-5 treatment stimulated a slight increase in the incorporation of glucose into glycogen, and, without being limited to any one theory, this may be due to its effects on the expression levels of glycogen synthase. Dkk-5 may also exert its effects on glucose metabolism in muscle by affecting the expression levels of proteins in the insulin-signaling pathway. Additionally, it is likely that Dkk-5 also affects the activity of proteins in the insulin-signaling pathway and/or regulates the translocation of the insulin-inducible glucose transporter (GLUT-4) in L6 cells.

In adipocytes, Dkk-5 treatment increased both basal and insulin-stimulated glucose uptake and the incorporation of glucose into lipids following 96-hr treatment. Glucose uptake and lipid accumulation in adipocytes depend on the differentiation state of the cells, and adipocyte differentiation is regulated by Wnt signaling. It is expected that active Dkk-5-overexpressing mice have enhanced glucose tolerance.

### Conclusion

Dkk-5 affected glucose metabolism in L6 muscle cells and is expected to do the same in transgenic mice overexpressing the protein in muscle using an expression system similar to the one above. Use of injected recombinant Dkk-5 protein preparation as set forth in the gel of FIG. 7 containing both the full-length and the 16-kDa portion thereof or injected 16-kDa portion alone is also expected to work to treat insulin resistance in mammals. Treatment of muscle cells with Dkk-5 (both full-length and internally cleaved 16-kDa product) resulted in an increase in the basal and insulin-stimulated glucose uptake. This effect was observed following long-term treatment, suggesting, without being limited to any one theory, that Dkk-5 may affect muscle differentiation and both the activity as well as the expression levels of proteins in the insulin-signaling pathway. The above observations demonstrate that Dkk-5 induces insulin sensitivity. Insulin resistance is a key feature of most forms of NIDDM. Hence, Dkk-5 would be useful in treating insulin-resistant disorders, and Dkk-5 is useful as a diagnostic marker in assays for such conditions. Also, Dkk-5 is expected to inhibit the progression of the diabetes phenotype in transgenic animal models, as disclosed, for example, in U.S. Pat. No. 6,187,991, and to be useful both in identifying new drugs to treat insulin-resistant disorders and in gene therapy using the techniques set forth in Larcher et al., supra, Ueki et al., supra, and Otaegui et al., supra.

### EXAMPLE 2

### Development of Anti-Dkk-5 Monoclonal Antibodies

Five female Balb/c mice (Charles River Laboratories, Wilmington, Del.) were hyperimmunized with purified recombinant polyhistidine-tagged (HIS8) human Dkk-5 expressed in baculovirus-infected insect cells (prepared as referenced in Example 1) and diluted in RIBI™ adjuvant (Ribi Immunochem Research, Inc., Hamilton, Mo.). The animals were immunized twice per week, with 50 μl used for each animal, administered via footpad. After five injections, B-cells from the lymph nodes of the five mice, demonstrating high anti-Dkk-5 antibody titers, were fused with mouse myeloma cells (X63.Ag8.653; American Type Culture Collection, Manassas, Va.) using the protocols described in Kohler and Milstein, supra, and Hongo et al., Hybridoma, 14: 253-260 (1995). After 10-14 days, the supernatants were harvested and screened for antibody production by direct ELISA. Seven positive clones, showing the highest immunobinding after the second round of subcloning by limiting dilution, were injected into PRISTANE™-primed mice (Freund and Blair, J. Immunol., 129: 2826-2830 (1982)) for in vivo production of the monoclonal antibodies. The ascites fluids were pooled and purified by Protein A affinity chromatography (PHARMACIA™ fast-protein liquid chromatography [FPLC]; Pharmacia, Uppsala, Sweden) as described by Hongo et al., supra. The purified antibody preparations were sterile filtered (0.2-elm pore size; Nalgene, Rochester N.Y.) and stored at 4° C. in phosphate-buffered saline (PBS).

These antibodies, prepared from the deposited hybridomas set forth below, can be used in the diagnostic methods set forth herein using the techniques described above.

Deposit of Material

The following materials have been deposited with the American Type Culture Collection, 10801 University Blvd., Manassas, Va. 20110-2209, USA (ATCC):

| Designation | ATCC Dep. No. | Deposit Date |
|---|---|---|
| DKK5.MAB3060.7A9.1A1.2G5 | PTA-3090 | Feb. 21, 2001 |
| DKK5.MAB3058.13E10.1G4.2B8 | PTA-3091 | Feb. 21, 2001 |
| DKK5.MAB3059.3A4.1B10.1G8 | PTA-3092 | Feb. 21, 2001 |
| DKK5.MAB3057.6C5.2C2.2E3 | PTA-3093 | Feb. 21, 2001 |
| DKK5.MAB3063.11A8.2F1.2B8 | PTA-3094 | Feb. 21, 2001 |
| DKK5.MAB3061.11H3.2F6.1E3 | PTA-3095 | Feb. 21, 2001 |
| DKK5.MAB3056.7H4.1H6.2B3 | PTA-3096 | Feb. 21, 2001 |

This deposit was made under the provisions of the Budapest Treaty on the International Recognition of the Deposit of Microorganisms for the Purpose of Patent Procedure and the Regulations thereunder (Budapest Treaty). This assures maintenance of a viable culture of the deposit for 30 years from the date of deposit. The deposit will be made available by ATCC under the terms of the Budapest Treaty, and subject to an agreement between Genentech, Inc. and ATCC, which assures permanent and unrestricted availability of the progeny of the culture of the deposit to the public upon issuance of the pertinent U.S. patent or upon laying open to the public of any U.S. or foreign patent application, whichever comes first, and assures availability of the progeny to one determined by the U.S. Commissioner of Patents and Trademarks to be entitled thereto according to 35 USC section 122 and the Commissioner's rules pursuant thereto (including 37 CFR section 1.14 with particular reference to 8860G 638).

The assignee of the present application has agreed that if a culture of the materials on deposit should die or be lost or destroyed when cultivated under suitable conditions, the materials will be promptly replaced on notification with another of the same. Availability of the deposited materials is not to be construed as a license to practice the invention in contravention of the rights granted under the authority of any government in accordance with its patent laws.

The foregoing written specification is considered to be sufficient to enable one skilled in the art to practice the invention. The present invention is not to be limited in scope by the constructs deposited, since the deposited embodiment is intended as a single illustration of certain aspects of the invention and any constructs that are functionally equivalent are within the scope of this invention. The deposit of material herein does not constitute an admission that the written description herein contained is inadequate to enable the practice of any aspect of the invention, including the best mode thereof, nor is it to be construed as limiting the scope of the claims to the specific illustrations that it represents. Indeed, various modifications of the invention in addition to those shown and described herein will become apparent to those skilled in the art from the foregoing description and fall within the scope of the appended claims.

The principles, preferred embodiments and modes of operation of the present invention have been described in the foregoing specification. The invention that is intended to be protected herein, however, is not to be construed as limited to the particular forms disclosed, since they are to be regarded as illustrative rather than restrictive. Variations and changes may be made by those skilled in the art without departing from the spirit of the invention.

1-31. (canceled)

32. A method of increasing cellular uptake of glucose, the method comprising administering Dickkopf-5 (Dkk-5) to the cells.

33. The method of claim 32, wherein said Dkk-5 comprises a polypeptide having the amino acid sequence of SEQ ID NO: 8.

34. The method of claim 32, wherein said Dkk-5 comprises a polypeptide having at least 85% identity to the amino acid sequence of residues 30 to 347 of SEQ ID NO: 5.

35. The method of claim 32, wherein said Dkk-5 comprises a polypeptide having at least 85% identity to the amino acid sequence of residues 25 to 347 of SEQ ID NO: 5.

36. The method of claim 32, wherein said Dkk-5 comprises a polypeptide having at least 85% identity to the amino acid sequence of residues 20 to 347 of SEQ ID NO: 5.

37. The method of claim 32, wherein said Dkk-5 comprises a polypeptide having the amino acid sequence of SEQ ID NO: 5.

38. The method of claim 32, further comprising administering to the cells an agent for treating insulin-resistance.

39. The method of claim 38, wherein the agent is insulin, IGF-1, or a sulfonylurea.

40. A method of increasing the cellular incorporation of glucose into glycogen, the method comprising administering Dickkopf-5 (Dkk-5) to the cells.

41. The method of claim 40, wherein said Dkk-5 comprises:
a) the amino acid sequence of SEQ ID NO: 8,
b) an amino acid sequence having at least 85% identity to the amino acid sequence of residues 30 to 347 of SEQ ID NO:5,
c) an amino acid sequence having at least 85% identity to the amino acid sequence of residues 25 to 347 of SEQ ID NO:5,
d) an amino acid sequence having at least about 85% identity to the amino acid sequence of residues 20 to 347 of SEQ ID NO:5; or
e) the amino acid sequence of SEQ ID NO:5.


SEQUENCE LISTING (8 sequences)

SEQ ID NO: 1 - protein, 266 aa, Homo sapiens
MMALGAAGATRVFVAMVAAALGGHPLLGVSATLNSVLNSNAIKNLPPPLGGAAGHPGSAVSAAPGILYPGGNKYQTIDNYQPYPCAEDEECGTDEYCASPTRGGDAGVQICLACRKRRKRCMRHAMCCPGNYCKNGICVSSDQNHFRGEIEETITESFGNDHSTLDGYSRRTTLSSKMYHTKGQEGSVCLRSSDCASGLCCARHFWSKICKPVLKEGQVCTKHRRKGSHGLEIFQRCYCGEGLSCRIQKDHHQASNSSRLHTCQRH

SEQ ID NO: 2 - protein, 259 aa, Homo sapiens
MAALMRSKDSSCCLLLLAAVLMVESSQIGSSRAKLNSIKSSLGGETPGQAANRSAGMYQGLAFGGSKKGKNLGQAYPCSSDKECEVGRYCHSPHQGSSACMVCRRKKKRCHRDGMCCPSTRCNNGICIPVTESILTPHIPALDGTRHRDRNHGHYSNHDLGWQNLGRPHTKMSHIKGHEGDPCLRSSDCIEGFCCARHFWTKICKPVLHQGEVCTKQRKKGSHGLEIFQRCDCAKGLSCKVWKDATYSSKARLHVCQKI

SEQ ID NO: 3 - protein, 350 aa, Homo sapiens
MQRLGATLLCLLLAAAVPTAPAPAPTATSAPVKPGPALSYPQEEATLNEMFREVEELMEDTQHKLRSAVEEMEAEEAAAKASSEVNLANLPPSYHNETNTDTKVGNNTIHVHREIHKITNNQTGQMVFSETVITSVGDEEGRRSHECIIDEDCGPSMYCQFASFQYTCQPCRGQRMLCTRDSECCGDQLCVWGHCTKMATRGSNGTICDNQRDCQPGLCCAFQRGLLFPVCTPLPVEGELCHDPASRLLDLITWELEPDGALDRCPCASGLLCQPHSHSLVYVCKPTFVGSRDQDGEILLPREVPDEYEVGSFMEEVRQELEDLERSLTEEMALGEPAAAAAALLGGEEI

SEQ ID NO: 4 - protein, 223 aa, Homo sapiens
MVAAVLLGLSWLCSPLGALVLDFNNIRSSADLHGARKGSQCLSDTDCNTRKFCLQPRDEKPFCATCRGLRRRCQRDAMCCPGTLCVNDVCTTMEDATPILERQDEQDGTHAEGTTGHPVQENQPKRKPSIKKSQGRKGQEGESCLRTFDCGPGLCCARHRWTKICKPVLLEGQVCSRRGHKDTAQAPEIFQRCDCGPGLLCRSQLTSNRQHARLRVCQKIEKL

SEQ ID NO: 5 - protein, 347 aa, Homo sapiens
MAGPAIHTAPMLFLVLLLPQLSLAGALAPGTPARNLPENHIDLPGPALWTPQASHHRRRGPGKKEWGPGLPSQAQDGAVVTATRQASRLPEAEGLLPEQSPAGLLQDKDLLLGLALPYPEKENRPPGWERTRKRSREHKRRRDRLRLHQGRALVRGPSSLMKKAELSEAQVLDAAMEESSTSLAPTMFFLTTFEAAPATEESLILPVTSLRPQQAQPRSDGEVMPTLDMALFDWTDYEDLKPDGWPSAKKKEKHRGKLSSDGNETSPAEGEPCDHHQDCLPGTCCDLREHLCTPHNRGLNNKCFDDCMCVEGLRCYAKFHRNRRVTRRKGCVEPETANGDQGSFINV

SEQ ID NO: 6 - protein, 12 aa, Homo sapiens
MAGPAIHTAPML

SEQ ID NO: 7 - protein, 8 aa, Homo sapiens
GALAPGTP

SEQ ID NO: 8 - protein, 13 aa, Homo sapiens
MALFDWTDYEDLK